(19) United States
(12) Patent Application Publication
(10) Pub. No.: US 2007/0048360 A1
(43) Pub. Date: Mar. 1, 2007

(54) Pharmaceutical compositions with melting point depressant agents and method of making same

(76) Inventors: Dario Norberto R. Carrara, Oberwil (CH); Arnaud Grenier, Steinbrunn le Haut (FR); Ingo Alberti, Huningue (FR); Christelle Rogue, Bouxwiller (FR); Celine Besse, Saint Louis (FR); Henry Laetitia, Saint Louis (FR)

Correspondence Address: WINSTON & STRAWN LLP;PATENT DEPARTMENT, 1700 K STREET, N.W., WASHINGTON, DC, 20006, US

(21) Appl. No.: 11/506,368

(22) Filed: Aug. 17, 2006

Related U.S. Application Data
(60) Provisional application No. 60/710,959, filed on Aug. 23, 2005.

(51) Int. Cl. A61K 9/70; A61K 9/48

(52) U.S. Cl. 424/443; 424/451; 424/449

(57) ABSTRACT

The invention relates to the use of chemical fragrance ingredient to lower the melting point of active agents, thereby changing crystalline active agents into an amorphous state. The invention also relates to methods of enhancing the transdermal or transmucosal skin permeation or skin penetration of pharmacologically active agents to patients in need thereof. The compositions of the present invention present the additional benefits of being substantially alcohol-free and having a pleasant olfactory profile.

This application claims the benefit of provisional application 60/710,959 filed Aug. 23, 2005, the entire content of which is expressly incorporated herein by reference.

### FIELD OF INVENTION

The present invention relates to novel topical compositions for transdermal or transmucosal delivery of pharmacologically active agents to a subject in need thereof. In particular, the invention relates to an alcohol-free or a substantially alcohol-free topical composition comprising a permeation enhancer comprising phenyl ethyl salicylate to enhance penetration of an active agent across mammalian dermal and/or mucosal surfaces. The invention also relates to a topical composition comprising a substantially amorphous pharmacologically active agent and a chemical fragrance ingredient, such as phenyl ethyl salicylate, and methods for making the same.

### BACKGROUND OF THE INVENTION

Transdermal delivery, i.e. the ability to deliver pharmaceuticals agents into and through skin surfaces, provides many advantages over oral or parenteral delivery techniques. In particular, transdermal delivery provides a safe, convenient and non invasive alternative to traditional administration systems that can provide a straightforward dosage regimen, relatively slow release of the drug into a patient's system, and control over blood concentrations of the drug. In contrast to oral administration, transdermal delivery typically does not produce the plasmatic peaks and valleys created by oral delivery and G.I. tract absorption. Second, transdermal delivery causes no gastrointestinal irritation, does not present restrictions around the time that the drug should be administered or whether or not the patient may eat afterwards. In particular, once-a-day transdermal delivery offers ease of use and is convenient, without the requirement to remember to take a drug at a specific time. Third, transdermal delivery improves patient compliance for patients who cannot swallow medication, for drugs with unpleasant taste and/or undergoing significant metabolism in the liver; the resulting increased bio-availability, which means that smaller doses may be used for the same drug, is responsible for minimized side effects. In contrast to parenteral administration, transdermal delivery typically does not cause pain and/or anxiety associated with needles, and does not present the risk of introducing infection to treated individuals, the risk of contamination or infection of health care workers caused by accidental needle-sticks and the risk of disposal of used needles.

The advantage of transdermal delivery is particularly enhanced in case of hydrophilic drugs, because of the molecular nature of the G.I. tract. As a lipid membrane, the G.I. tract possesses hydrophobic properties, thus the more hydrophilic a drug is, and the more likely it is to be absorbed poorly through the G.I. tract. A well known example of this problem is sodium alendronate, a bisphosphonate, which needs to be administered in very large doses because only a very small fraction of the drug (about 0.6) % is absorbed indeed when administered orally (please refer to FOSAMAX® Tablets and Oral Solutions Prescribing Information, issued by Merck & Co., Inc.).

However, despite its clear advantages, transdermal delivery also poses inherent challenges, in part because of the nature of skin. Skin is essentially a thick membrane that protects the body by acting as a barrier. Consequently, passive delivery through intact skin necessarily entails the transport of molecules through a number of structurally different tissues, including the stratum corneum, the viable epidermis, the papillary dermis and the capillary walls in order for the drug to gain entry into the blood or lymph system. Each tissue features a different resistance to penetration, but the stratum corneum is the strongest barrier to the absorption of transdermal and topical drugs. The tightly packed cells of the stratum corneum are filled with keratin. The keratinization and density of the cells may be responsible for skin's impermeability to certain drugs. Transdermal delivery systems must therefore be able to overcome the various resistances presented by each type of tissue.

Transdermal delivery is different from topical delivery. Drugs administered transdermally are absorbed through skin or mucous membranes and provide effects beyond the application site. In contrast, purpose of a topical drug, e.g., antibiotic ointment, anti-acne cream, hair-growing lotion, anti-itching spray, is to administer medication at the site of intended action. Topical medications typically should be designed not to permit significant drug passage into the patient's blood and/or tissues. Topical formulations are often used to treat infections or inflammations. They also are used as cleansing agents, astringents, absorbents, keratolytics, and emollients. The vehicle of a topical treatment, i.e. the non-active component(s) that carries the active ingredient(s), may interact with the active ingredient(s), changing the drug's effectiveness. The vehicle may also cause skin irritation or allergic reactions in some patients. Thus, the vehicle must be selected with extreme care. Topical formulations may be prepared as pastes, gels, creams, ointments, lotions, solutions, or aerosols. Occlusion with household plastic wrap, bandages, plasters, or plastic tape, is often used in conjunction with topical treatments to improve the drug's absorption and its effectiveness. Typically non-occlusive dosage forms are applied to the skin or mucosa and are left uncovered and open in the atmosphere.

In recent years, advances in transdermal and topical delivery include the formulation of skin penetration enhancing agents, also known as permeation enhancers. Permeation enhancers are often lipophilic chemicals that readily move into the stratum corneum and enhance the movement of drugs through the skin. Energy-assisted skin permeation techniques also have emerged to improve transdermal delivery, including heat, ultrasound, iontophoresis, and electroporation. But even with these methodologies, only a limited number of drugs can be administered transdermally without problems such as sensitization or irritation occurring.

The inefficiencies of drug permeation across or through the skin or mucosa barriers are known. It is also known that the permeation of a drug in a non-occlusive transdermal or transmucosal dosage form can be as little as 1% and usually is no more than 15%. Thus, a vast majority of the active drug remains unabsorbed on the skin or mucosa surface, resulting in a low bioavailability of the particular drug, and also in a high risk of contamination of other individuals in close proximity to the user is presented by the unwanted transfer of the pharmaceutical formulation in the non-occlusive dosage form.

Transdermal delivery of a drug or active agent conventionally requires that the drug be presented to the absorption barrier, e.g., skin, in a lipophilic form in solution. This requires very often the use of organic solvents comprising, but not limited to, short-chain alcohol (ethanol, propanol, isopropanol, butanol), glycols (propylene glycol, polyethylene glycols), glycol ethers, N-methyl-pyrrolidone, 2-pyrrol, dimethyl isosorbide. Such organic solvents are also well known for causing local skin reactions, such as dryness, redness, itching, stinging, burning, erythema, the importance of which is dependent on (i) the amount of solvent applied on the skin and (ii) the frequency of application and (iii) the extent of the surface of application. Use of such organic solvents is even more a concern when considering administration of active drugs through mucosal surfaces, such as the ocular mucosa, the nasal mucosa, the buccal mucosa, the rectal mucosa and the vaginal mucosa. In addition, the drug levels in solution should be as close as possible to saturation, to provide the highest possible concentration gradient, the “driving force” (also referred as thermodynamic activity of the drug) for permeation of said drug across the absorption barrier. However, though maximal thermodynamic activity (equal to 1) is obtained from drug crystals, crystallization of drugs must be prevented since the solid crystals can not permeate spontaneously through a biological membrane. Hence the recourse to solvents and co-solvents is necessary to maintain the drug as close to saturation as possible.

Conventionally, a solution of a drug in a lipophilic form is achieved by including either a water miscible co-solvent or an emulsified oil phase in which the drug is first dissolved in an oil or mixture of oils. Both of these techniques hinder drug penetration by providing a competing phase for drug migration across the barrier, however, and the negative effect of an emulsified oil phase is more pronounced. Further, attempts to overcome this drawback with the use of organic co-solvents, such as the ones cited herein above, is known to cause adverse local reactions on the skin and epithelia. Thus, there have been many attempts to improve the formulations for transdermal and topical pharmacological compositions to enhance patient comfort, efficiency, absorption, and bioavailability.

One attempt to improve transdermal or transmucosal absorption of active drugs from pharmacological formulations is the decrease of melting point of said active drugs. Decrease of melting point of an active drug presents numerous advantages, including, but not limited to, the increase of the solubility of a compound (see Hadgraft in “Transdermal Delivery: Present and Future Perspectives”, The Drug Delivery Reports Company Spring/Summer 2003, © PharmaVentures Ltd 2003, http://www.ddcr.com/articles/ddcr_s2003_article3.pdf), and the increase of its permeability through the skin and the mucosa membranes (see Guy and Hadgraft, “Transdermal Drug Delivery”, Marcel Dekker, 1989, presented hereinafter in “Detailed Description of Preferred Embodiments”). Thus decrease of melting point of an active drug, if possible, would alleviate partially or totally the recourse to organic solvents as explained herein before.

Decrease of melting point of active drugs may be achieved by specific selection of drug enantiomers: see, for instance, U.S. Pat. No. 5,114,946.

Decrease of melting point of active drugs may also be achieved by formation of so-called eutectic mixtures. Eutectic mixtures are defined as “the point on a two-component solid-liquid phase diagram which represents the lowest melting point of any possible mixture.” (“Handbook of Chemistry and Physics”, 79th ed., David R. Lide, CRC Press LLC, 1998). A eutectic mixture of two eutectic-forming solids shows, upon intimate admixture of the two solids, a homogeneous liquid phase above the melting point of the higher melting component. Usually, although not always, the required intimate mixture involves melting the two eutectic-forming solids together. A plot of melting point versus relative composition of the two eutectic-forming solids displays a minimum point between two intersecting lines at which a homogeneous liquid phase coexists with each of the respective homogeneous solid phases. This point is known as the eutectic point or eutectic temperature, and is represented by point E in the following diagram below. As shown in the diagram below, the melting temperatures of two substances (A and B) are plotted against mixture composition. Upon addition of B to A, or of A to B, melting points are reduced. At a particular composition (the eutectic mixture composition), the eutectic point is reached that represent the lowest melting point of any mixture of A and B. Below the eutectic temperature, no liquid phase exists. If the solution of A and B is cooled which is richer in A than the eutectic mixture, crystals of pure A will appear. As the solution is cooled further, more and more A crystallizes out and the solution becomes richer in B. When the eutectic point is reached, the remaining solution crystallizes out forming a microcrystalline mixture of pure A and pure B. The administration of a eutectic mixture composed of a drug and a substance readily soluble in water has been used in pharmaceuticals. The soluble carrier dissolves leaving the drug in a fine state that will rapidly go into solution.

[image]

Thus, a liquid having a eutectic composition freezes at a single temperature without change of composition. Because they enable use of lower temperatures during formation of solders, and are intimate admixtures of conductors, eutectic mixtures are known and used in the metals and alloys industry as well as in the electronics, where the formation of lower melting point eutectic mixtures is generally regarded as advantageous. Another very well known application of eutectic mixtures is the sawing of sodium chloride (NaCl) on roads in winter to prevent the formation of ice: when exact proportions are met (about 77% w/w of sodium chloride), water and NaCl form a pure eutectic mixture whose melting point is decreased as low as −21° C. When NaCl (Na⁺, Cl⁻) enters into contact with ice, ions re-organized themselves around the polar molecules of water (H₂^{δ+}, O^{δ-}) and form a “new” compound: (H₂O).(NaCl); this re-organization only requires that atoms move slightly, and therefore are possible in solid phase. Re-organization of water and NaCl can only take place at contact surfaces between ice crystals and NaCl, i.e. at the ice surface. Therefore a thin, superficial eutectic layer forms and melts (provided temperature is above −21° C.). Since NaCl is present as supersaturated state, it dissolves within the molten eutectic, and is then able to react with further ice crystals. This phenomenon propagates until either NaCl or water is missing.

While use of eutectic mixtures in pharmaceutical formulations has been contemplated, it has not been widely utilized because of the perceived problems associated with such use. For example, it is believed that eutectic formation in common pharmaceutical dosage forms is undesirable and can be prevented, for example, by the use of an inert diluent such as lactose in sufficient quantity to prevent intimate contact between the eutectic-forming solid components. See, “Pharmaceutical Dosage Forms and Drug Delivery Systems”, 8^{th} ed. (Ansel, H. C., Popovich, N. G. and Allen, L. V. Jr., p. 194, 2005). Moreover, U.S. Pat. No. 5,512,300, which is incorporated herein by reference, discloses that the formation of eutectic mixtures results in stability problems in solid dosage forms and is, therefore, to be avoided. That patent further teaches a method of preventing formation of such mixtures by alkali metal treatment.

U.S. Pat. Nos. 4,529,601 and 4,562,060 to Broberg et al., disclose topical compositions containing eutectic compositions and methods of local anesthesia by administering on the skin specific combination of local anesthetics in preferred ratios. The most preferred eutectic mixture is a lidocaine:prilocalne mixture in a 1 to 1 ratio, from which EMLA®, the only commercially available pharmaceutical drug product comprising a eutectic mixture, has been developed.

The ability of menthol to form eutectic mixture with some active drugs is also well known in the art. See, for instance, P. W. Stott, A. C. Williams, and B. W. Barry: “Transdermal delivery from eutectic systems: enhanced permeation of a model drug, ibuprofen.”, in Journal of Controlled Release, 50:297-308, 1998. See also Kang L, Jun H W, McCall J W, “Physicochemical studies of lidocaine menthol binary systems for enhanced membrane transport”, Int J Pharm. 2000, sep 25; 206(1-2):35-42. However, such eutectic mixtures are obtained at ratio of active drug:menthol 30:70, and the very high amounts of menthol required (about 12% for a 5% ibuprofen formulation for instance) would cause discomfort to the patient (very strong, unpleasant smell, unpleasant exaggerated cooling sensation upon application, rubefaciant action, and local skin irritation). See also Touitou et al., in “Testosterone skin permeation enhancement by menthol through formation of eutectic with drug and interaction with skin lipids”, J. Pharm. Sci. 1997, 86, 1394-1399.

U.S. Pat. No. 6,368,618 to Jun et al., discloses topical compositions comprising nonsteroidal anti-inflammatory drug(s); at least one melting point depressing agent selected from the group of terpenes (namely, thymol, menthol, eucalyptol, or eugenol), a group of active drugs (methyl salicylate, phenyl salicylate, capsaicin, or a local anesthetic agent such as lidocaine) or a group of antioxidants (butylated hydroxytoluene), and any combination thereof; and at least one alcohol. However, eugenol is listed as a potential fragrance allergen by the European Community. Furthermore, terpenes, such as thymol, menthol, eucalyptol, limonene, citronellol, geraniol, are known to be skin irritant. The U.S. Pat. No. 6,368,618 patent also discloses the synergistic combination of an alcohol with a melting point depression agent to observe a significant melting point depression. This patent further teaches that preferred nonsteroidal anti-inflammatory drugs are chiral compounds present as a substantially pure stereoisomer.

U.S. Pat. No. 6,410,036 to De Rosa et al., discloses cosmetic compositions comprising eutectic mixture of hydroxyl acids and carbohydrates, polyols, amino acids or carboxylic acids.

U.S. Pat. No. 6,841,161 to Passmore et al., discloses a composition comprising a eutectic mixture of at least two pharmacologically active agents in their lipophilic (substantially water-insoluble) form for mutual enhancement of transdermal permeation. The requirement for use of at least two pharmacologically active agents, however, is disadvantageous in requiring the use of multiple active agents. The second pharmacological agent used in the composition would in some cases have a completely different therapeutic effect than the first pharmacological agent, and may be non desirable and/or may not be medically efficient or practical.

It is hypothesized that increased thermodynamic activity and resulting increased drug flux observed for eutectic compositions is due to the amorphous nature of the eutectic, i.e. to the inhibition of crystallization in the eutectic system (see Santos et al., “Transdermal Delivery of Ibuprofen Using Microemulsions and Eutectic Systems”, Controlled Release Society Symposium, Jul. 22-26, 2006, Vienna, Austria). Temporary amorphous pharmaceutical compositions from volatile solvent-based vehicles are described by in U.S. Pat. No. 4,820,724 or by Feldmann et al. in “Percutaneous penetration of 14C hydrocortisone in man. II. Effect of certain bases and pre-treatment”, Arch. Derm., 94, 649-651, 1966). However, such systems require large amounts of alcohols and organic solvents, such as ethanol and acetone, which may be irritant for the skin.

In view of the foregoing, it appears obvious that there is a need for transdermal and topical compositions presenting enhanced drug permeation properties for a wide variety of pharmaceutically active agents.

There is another need for transdermal and topical compositions wherein presence of multiple pharmaceutical agents is not required to promote permeation of one or all of said pharmaceutical agent(s).

There is another further need for transdermal and topical compositions of pharmaceutical agent(s) wherein presence of significant amounts of organic solvents is not required to maintain said pharmaceutical agent(s) in a state compatible with permeation through or penetration to the skin or the mucosa surfaces.

There is another further need for transdermal and topical compositions of pharmaceutical agent(s) wherein crystallization of said pharmaceutical agent(s) is significantly delayed or even totally prevented without requiring the use of high amounts of organic solvents and co-solvents.

There is another further need for transdermal and topical compositions of pharmaceutical agent(s) with improved patient compliance, e.g. for instance having a pleasant olfactory profile, being free or substantially free of alcohols responsible for skin dryness, redness, itching, and/or being devoid of skin irritation potential.

The present invention described herein after addresses all of the aforementioned needs by providing aqueous transdermal and topical compositions containing amorphous or substantially amorphous pharmaceutical active agents whose melting point is depressed thanks to specific combinations with chemical fragrance and flavor ingredients.

No admission is made that any reference, including any patent or patent document, cited in this specification constitutes prior art. In particular, it will be understood that, unless otherwise stated, reference to any document herein does not constitute an admission that any of these documents forms part of the common general knowledge in the art in United States of America or in any other country. The discussion of the references states what their authors assert, and the applicant reserves the right to challenge the accuracy and pertinency of the documents cited herein.

### SUMMARY OF THE INVENTION

The present invention generally relates to improved transdermal and topical pharmacological compositions.

In one aspect of the invention, the composition comprises an active agent and a chemical fragrance or flavor ingredient (“CFI”), wherein the melting point of said active agent is depressed by the CFI. The active agent and the CFI are each present in an amount sufficient to form an amorphous or substantially amorphous liquid or semi-solid state of said active agent. The CFI includes, but is not limited to, phenyl ethyl salicylate, 4-(1,3-benzodioxol-5-yl)butan-2-one, β-naphtyl isobutyl ether, indeno-m-dioxin tetrahydro, ortho tertiary butyl cyclohexanol, and the like, the chemical structures of which are included herein after.

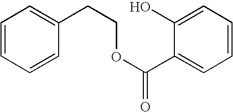

Phenyl ethyl salicylate (CAS # 887-22-9)
Log P: 4.31
Melting point: 39° C.-43° C.
Olfactory description: rosy character, very sweet but mild and balsamic

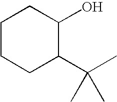

Ortho tertiary butyl cyclohexanol (CAS # 13491-79-7)
Log P: 3.3-3.4
Melting point: about 45° C.
Olfactory description: extremely powerful chemical with a minty,
camphoraceous odor in the pine, patchouli families

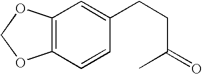

4-(1,3-benzodioxol-5-yl)butan-2-one (CAS # 55418-52-5)
Log P: 1.15
Melting point: about 47° C.-50° C.
Olfactory description: extremely sweet odor with raspberry, cotton candy,
cassis notes

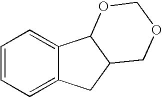

Indeno-m-dioxin tetrahydro (CAS # 18096-62-3)
Log P: 1.33
Melting point: 36° C.-40° C.
Olfactory description: floral, jasmine note

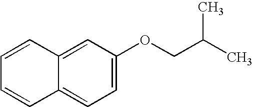

β-naphtyl isobutyl ether (CAS # 2173-57-1)
Melting point: 32° C.-34° C.
Olfactory description: sweet tenacious fruity and floral note; reminiscent
neroli type odor; intensely fruity strawberry type taste in dilution.

In another aspect of the invention, the transdermal or topical composition comprising an active agent and a CFI, unlike conventional transdermal or topical compositions which require the presence of alcohol for permeation through the skin, can be substantially alcohol-free. Accordingly, the adverse effects of including alcohol in a transdermal or topical composition can be minimized or eliminated.

In another aspect of the invention, the transdermal or topical composition comprising an active agent and a CFI, the composition provides enhanced transdermal or transmucosal permeation and/or drug flux of said active agent compared to transdermal or topical compositions not containing a mixture of an active agent and a CFI.

In another aspect of the invention, advantageously the transdermal or topical composition comprising an active agent and a CFI does not require incorporation of further inactive ingredients to impart a pleasant odor profile to said composition.

In another aspect of the invention, the permeation of a variety of active agents can be improved by CFI, as will be discussed herein below. The active agent can be selected from the group comprising, but not limited to, ibuprofen, ketoprofen, lidocaine, prilocalne, bupivacaine, procaine, fentanyl, benzoyl peroxide, captopril, carmustin, carvedilol, chlorpromazine, clonidine, ephedrine, granisetron, nicotine, oxybutynin, ropinirole, pramipexole, promethazine, propranolol, scopolamine, and testosterone. In addition, none of the melting point depressant agents of the present invention are subject to EU Fragrance Allergen labeling.

In accordance with one embodiment of the invention, the topical composition further includes a pharmaceutically acceptable carrier, and the composition is in the form of an emulsified gel (or gellified emulsion). The emulsion includes a discontinuous phase and a continuous phase. The discontinuous phase includes the amorphous, non-solid mixture of the active agent and the CFI. The continuous phase includes the pharmaceutically acceptable carrier. The continuous phase may further include an emulsifying agent or an emulsifying system, and a thickening agent or a thickening system.

In yet another aspect of the invention, a method for preparing a topical composition for enhanced transdermal or transmucosal delivery of a pharmacologically active agent is provided. The method comprises forming an amorphous, non-solid mixture which includes a pharmacologically active agent and a CFI; and associating said mixture with a pharmaceutically acceptable carrier, such that the composition provides enhanced transdermal or transmucosal permeation of the pharmacologically active agent.

In yet another aspect of the invention, method can include at least two pharmacologically active agents. Advantageously, the at least two active agents are contained within a single common composition. However, the at least two active agents can be contained in two distinct compositions, which can then be dispensed from a single common dispenser either simultaneously or consecutively. In this manner, the dispenser preferably includes at least two separate compartments in which each active agent is maintained in the dispenser separately from the other active agent. The dispenser can have a single actuator for dispensing each of the at least two active agents. Alternatively, the dispenser can have a plurality of actuators for each compartment. If desired, the at least two active agents can remain separated until dispensing. A variety of different types of dispensers can be used. For example, the dispenser can be a metered dose pump, or a dispensing tube.

According to yet another aspect, the invention relates to a method for providing enhanced transdermal or transmucosal permeation of at least one pharmacologically active agent comprising administering an amount of a transdermal or topical composition comprising the pharmacologically active agent in an amorphous, non-solid mixture with at least one CFI to a subject in need thereof.

These and other embodiments of the present invention will readily occur to those of ordinary skill in the art in view of the disclosure herein.

### BRIEF DESCRIPTION OF THE DRAWINGS

The features and benefits of the invention will now become more clear from a review of the following description of illustrative embodiments and the accompanying drawings, wherein:

FIG. 1 is a graphic representation of the differential scanning calorimetry (DSC) thermogram of a pure chemical fragrance or flavor ingredient (CFI) in accordance with the present invention;

FIG. 2 is a graphic representation of the DSC thermogram of a pure active pharmaceutical ingredient (API) in accordance with the present invention;

FIG. 3 is a graphic representation of the DSC thermogram of a 83.3:16.7 API:CFI mixture in accordance with the present invention;

FIG. 4 is a graphic representation of the DSC thermogram of a 61.3:38.7 API:CFI mixture in accordance with the present invention;

FIG. 5 is a graphic representation of the DSC thermogram of a 56.6:43.4 API:CFI mixture in accordance with the present invention;

FIG. 6 is a graphic representation of the DSC thermogram of a 41.0:59.0 API:CFI mixture in accordance with the present invention;

FIG. 7 is a graphic representation of the DSC thermogram of a 24.2:75.8 API:CFI mixture in accordance with the present invention;

FIG. 8 is a graphic representation of the DSC thermogram of a 15.7:84.3 API:CFI mixture in accordance with the present invention;

FIG. 9 is a graphic representation of biodistribution of lidocaine through various layers of the skin;

FIG. 10 is a graphic representation of the DSC thermogram of pure ibuprofen;

FIG. 11 is a graphic representation of the differential scanning calorimetry (DSC) thermogram of a pure chemical fragrance or flavor ingredient (CFI) in accordance with the present invention;

FIG. 12 is a graphic representation of the DSC thermogram of a 50:50 API:CFI mixture in accordance with the present invention;

FIG. 13 is graphic representation of relative kinetic profiles of a formulation comprising a mixture of ibuprofen and phenyl ethyl salicylate in accordance with the present invention compared with a formulation comprising ibuprofen but which does not include phenyl ethyl salicylate; and

FIG. 14 is graphic representation of drug flux profiles of a formulation comprising a mixture of ibuprofen and phenyl ethyl salicylate in accordance with the present invention compared with a formulation comprising ibuprofen but which does not include phenyl ethyl salicylate; and

FIG. 15 is graphic representation of relative kinetic profiles of a formulation comprising a mixture of ketoprofen and phenyl ethyl salicylate in accordance with the present invention compared with a formulation comprising ketoprofen but which does not include phenyl ethyl salicylate; and

FIG. 16 is graphic representation of drug flux profiles of a formulation comprising a mixture of ketoprofen and phenyl ethyl salicylate in accordance with the present invention compared with a formulation comprising ketoprofen but which does not include phenyl ethyl salicylate.

FIG. 17 is a graphic representation of the DSC thermogram of pure oxybutynin;

FIG. 18 is a graphic representation of the DSC thermogram of a 53.8:46.2 API:CFI mixture in accordance with the present invention;

FIG. 19 is graphic representation of relative kinetic profiles of a formulation comprising a mixture of granisetron and phenyl ethyl salicylate in accordance with the present invention compared with a formulation comprising granisetron but which does not include phenyl ethyl salicylate; and

FIG. 20 is graphic representation of drug flux profiles of a formulation comprising a mixture of granisetron and phenyl ethyl salicylate in accordance with the present invention compared with a formulation comprising granisetron but which does not include phenyl ethyl salicylate.

### DETAILED DESCRIPTION OF PREFERRED EMBODIMENTS

It is to be understood that the terminology used herein is for the purpose of describing particular embodiments only, and is not intended to be limiting. As used in this specification, description of specific embodiments of the present invention, and any appended claims, the singular forms “a,” “an” and “the” include plural referents unless the context clearly dictates otherwise. Thus, for example, reference to “a cosolvent” includes two or more cosolvents, mixtures of cosolvents, and the like, reference to “a compound” includes one or more compounds, mixtures of compounds, and the like.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, the preferred materials and methods are described herein.

In describing and claiming the present invention, the following terminology will be used in accordance with the definitions set out below.

The phrase “dosage form” as used herein refers to a pharmaceutical composition comprising an active agent and optionally containing inactive ingredients, e.g., pharmaceutically acceptable excipients such as suspending agents, surfactants, disintegrants, binders, diluents, lubricants, stabilizers, antioxidants, osmotic agents, colorants, plasticizers, coatings and the like, that may be used to manufacture and deliver active pharmaceutical agents.

The phrase “gel” as used herein refers to a semi-solid dosage form that contains a gelling agent in, for example, an aqueous, alcoholic, or hydroalcoholic vehicle and the gelling agent imparts a three-dimensional cross-linked matrix (“gellified”) to the vehicle. The term “semi-solid” as used herein refers to a heterogeneous system in which one solid phase is dispersed in a second liquid phase.

The phrase “carrier” or “vehicle” as used herein refers to carrier materials (other than the pharmaceutically active ingredient) suitable for transdermal or topical administration of a pharmaceutically active ingredient. A vehicle may comprise, for example, solvents, cosolvents, permeation enhancers, pH buffering agents, antioxidants, gelling agents, preservatives, colorants, additives, or the like, wherein components of the vehicle are nontoxic and do not interact with other components of the total composition in a deleterious manner.

The phrase “non-occlusive transdermal or topical drug delivery” as used herein refers to transdermal delivery methods or systems that do not occlude the skin or mucosal surface from contact with the atmosphere by structural means, for example, by use of a patch device, a fixed application chamber or reservoir, a backing layer (for example, a structural component of a device that provides a device with flexibility, drape, or occlusivity), a tape or bandage, or the like that remains on the skin or mucosal surface for a prolonged period of time. Non-occlusive transdermal or topical drug delivery includes delivery of a drug to skin or mucosal surface using a topical medium, for example, creams, ointments, sprays, solutions, lotions, gels, and foams. Typically, non-occlusive transdermal drug delivery involves application of the drug (in a topical medium) to skin or mucosal surface, wherein the skin or mucosal surface to which the drug is applied is left open to the atmosphere.

The phrase “occlusive transdermal or topical drug delivery” as used herein refers to transdermal delivery methods or systems that occlude the skin or mucosal surface from contact with the atmosphere by structural means, for example, by use of a patch device, a fixed application chamber or reservoir, a backing layer (for example, a structural component of a device that provides a device with flexibility, drape, or occlusion), a tape or bandage, or the like that remains on the skin or mucosal surface for a prolonged period of time. Occlusive transdermal or topical drug delivery includes delivery of a drug to skin or mucosal surface using a topical medium, for example, creams, ointments, sprays, solutions, lotions, gels, and foams under occlusion. Typically, occlusive transdermal or topical drug delivery involves application of the drug (in a topical medium) to skin or mucosal surface, wherein the skin or mucosal surface to which the drug is applied is protected from the atmosphere.

The phrase “systemic” delivery, as used herein, refers to both transdermal (and “percutaneous”) and transmucosal administration, that is, delivery by passage of a drug through a skin or mucosal tissue surface and ultimately into the bloodstream.

The phrase “topical” delivery, as used herein, refers to delivery of a drug to any accessible body surface such as, e.g. for instance the skin, the nasal mucosa, the auricular mucosa, the buccal mucosa, the ocular mucosa, the pulmonary mucosa, the vaginal mucosa and rectal mucosa, as well as gastrointestinal epithelium, that is, penetration of a drug into a skin or mucosal tissue surface for local action.

The phrase “administration of active agents” as used herein can be understood to include local administration or systemic administration. For instance in case of the transdermal route, “administration of active agents” can be understood to include local penetration into the different layers of the skin or permeation through the skin into the systemic compartments.

The phrase “therapeutic agent”, “pharmaceutical agent”, “pharmacological active agent” or “active agent”, which are used interchangeably, as used herein, can be understood to include any substance or formulation or combination of substances or formulations of matter which, when administered to a human or animal subject, induces a desired pharmacologic and/or physiologic effect by local and/or systemic action.

The phrase “excipient” as used herein refers to any inert substance combined with an active agent to prepare a convenient dosage form and vehicle for delivering the active agent.

The phrase “therapeutically effective amount” as used herein refers to a nontoxic but sufficient amount of a drug, agent, or compound to provide a desired therapeutic effect.

The phrase “substantially” as used herein refers to an amount of a present ingredient, component or additive that is less than that which is necessary to impart the characteristics of the ingredient, component or additive to the composition.

The phrase “dose” and “dosage” as used herein refers to a specific amount of active or therapeutic agents for administration.

The phrase “chemical fragrance ingredient” or “chemical flavor ingredient” (CFI) as used herein refers to pure or substantially pure edible chemicals used in the pharmaceutical industry, or in the food industry, or in the cosmetic industry, or in the industry of household and toiletries, whose primary function is to emit a pleasant odor (or aroma, bouquet, perfume, redolence, scent) or flavor (or taste, savor) to a substance or a composition. Flavor is the sensory impression of a substance, and is determined mainly by the chemical senses of taste and smell. The “trigeminal senses,” which detect chemical irritants in the mouth and throat, may also occasionally determine flavor. The flavor of a substance, as such, can be altered with natural or artificial flavorants or fragrances, which affect these senses. While the taste (or flavor) of a substance is limited to sweet, sour, bitter, salty, umami, and other basic tastes, the fragrances of a substance are potentially limitless. For instance, a food's flavor can be easily altered by changing its smell while keeping its taste similar. For this reason the same terms are usually used in the fragrance and flavors industry to refer to edible chemicals and extracts that alter the flavor of compositions through the sense of smell. Therefore the phrase “chemical fragrance ingredient” or “chemical flavor ingredient” (CFI) as used herein are totally interchangeable.

The phrase “eutectic mixture” as used herein refers to any mixture of a CFI as previously defined and of an active agent whose melting point is lower than any of its single constituents. It will be appreciated that, unless specified otherwise, the phrase eutectic mixture as used herein also encompasses mixtures of an active agent and a CFI wherein melting point of said active agent is lowered in the presence of the CFI.

The phrase “amorphous” as used herein refers to substantially not crystallized. It will be appreciated that, unless specified otherwise, the phrase amorphous encompasses a certain degree of crystallinity, so that the ratio of non crystallized active drug to crystallized active drug is preferably superior to 1. Methods which may be used to assess ratio of non crystallized active drug to crystallized active drug include, but are not limited to, Differential Scanning Calorimetry or microscopy.

The phrase “solvent” refers herein to “volatile solvent” and “non-volatile solvents”. A volatile solvent is a solvent that changes readily from solid or liquid to a vapor, and that evaporates readily at normal temperatures and pressures. Examples of volatile solvents include, but are not limited to, ethanol, propanol, butanol, isopropanol, and/or mixtures thereof. A non-volatile solvent is a solvent that does not change readily from solid or liquid to a vapor, and that does not evaporate readily at normal temperatures and pressures. Examples of non-volatile solvents include, but are not limited to, propylene glycol, glycerin, liquid polyethylene glycols, polyoxyalkylene glycols, and/or mixtures thereof. Stanislaus, et al., (U.S. Pat. No. 4,704,406) defined “volatile solvent” as a solvent whose vapor pressure is above 35 mm Hg when skin temperature is 32° C., and a “non-volatile” solvent as a solvent whose vapor pressure is below 10 mm Hg at 32° C. skin temperature. Solvents used in the practice of the present invention are typically physiologically compatible and used at non-toxic levels.

The phrase “cosolvent” herein refers to water-miscible organic solvents that are used in liquid drug formulations to increase the solubility of poorly water-soluble substances or to enhance the chemical stability of a drug. The phrase “solvent” and “cosolvent” as used herein are totally interchangeable.

The phrase “alcohol” as used herein refers to a short-chain C₂-C₄ alcohol, for example, ethanol, propanol, butanol, isopropanol, propylene glycol, diethylene glycol mono ethyl ether, glycofurol, and/or mixtures of thereof.

The phrase “permeation enhancer” or “penetration enhancer” as used herein refers to an agent that improves the rate of transport of a pharmacologically active agent (e.g., nicotine) across the skin or mucosal surface. Typically a penetration enhancer increases the permeability of skin or mucosal tissue to a pharmacologically active agent. Penetration enhancers, for example, increase the rate at which the pharmacologically active agent permeates through skin and enters the bloodstream. Enhanced permeation effected through the use of penetration enhancers can be observed, for example, by measuring the flux of the pharmacologically active agent across animal or human skin as described in the Examples herein below. An “effective” amount of a permeation enhancer as used herein means an amount that will provide a desired increase in skin permeability to provide, for example, the desired depth of penetration of a selected compound, rate of administration of the compound, and amount of compound delivered.

The phrase “synergy”, “synergism”, “synergistic effect” or “synergistic action” as used herein means an effect of the interaction of the actions of two agents such that the result of the combined action is greater than expected as a simple additive combination of the two agents acting separately.

The phrase “effective” or “adequate” permeation enhancer or combination as used herein means a permeation enhancer or a combination that will provide the desired increase in skin permeability and correspondingly, the desired depth of penetration, rate of administration, and amount of drug delivered.

The phrase “thermodynamic activity” of a substance means the energy form involved in skin permeation of this substance. The chemical potential of a substance is defined in thermodynamics as the partial molar free energy of the substance. The difference between the chemical potentials of a drug outside and inside the skin is the energy source for the skin permeation process.

The phrase “stratum corneum” as used herein refers to the outer layer of the skin. The stratum corneum typically comprises layers of terminally differentiated keratinocytes (made primarily of the proteinaceous material keratin) arranged in a brick and mortar fashion wherein the mortar comprises a lipid matrix (containing, for example, cholesterol, ceramides, and long chain fatty acids). The stratum corneum typically creates the rate-limiting barrier for diffusion of the active agent across the skin.

The phrase “intradermal depot” as used herein refers to a reservoir or deposit of a pharmaceutically active compound within or between the layers of the skin (e.g., the epidermis, including the stratum corneum, dermis, and associated subcutaneous fat), whether the pharmaceutically active compound is intracellular (e.g., within keratinocytes) or intercellular.

The term “subject” as used herein refers to any warm-blooded animal, particularly including a member of the class Mammalia such as, without limitation, humans and non human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex.

Before describing the present invention in detail, it is to be understood that this invention is not limited to particular embodiments described herein, for example, particular solvent(s), antioxidant(s), cosolvent(s), penetration enhancer(s), buffering agent(s), preservative(s), and/or gelling agent(s), and the like, as use of such particulars may be selected in view of the teachings of the present specification by one of ordinary skill in the art. It is also to be understood that the terminology used herein is for the purpose of describing particular embodiments of the invention only, and is not intended to be limiting.

The present invention relates to a composition for enhanced transdermal or transmucosal permeation of a pharmacologically active agent. The topical composition of the invention can be in a semi-solid form such as, but not limited to, solutions, lotions, gels, creams and the like. Advantageously, the topical composition can be substantially alcohol-free, thereby minimizing or eliminating adverse local reactions. Alternatively, the composition can be in the form of an oral capsule.

In one embodiment of the present invention, the composition comprises an active agent in combination with a CFI, such as 4-(1,3-benzodioxol-5-yl)butan-2-one, β-naphtyl isobutyl ether, tetrahydro indeno-m-dioxin, or phenyl ethyl salicylate, and mixtures thereof. In this regard, it has surprisingly been found that particular CFI compound can decrease melting point of particular active agents. Ratios of the active agent to the CFI range from 90:10 to 10:90. Preferred ratios are ratios wherein the mixture contains more active agent than the CFI, e.g. for instance ratios ranging from 90:10 to 50:50. Melting point of active agents is preferably decreased down to ambient temperature or below, i.e. typically down to 25° C. or below. A preferred CFI is PES.

In another embodiment of the present invention, the intimate mixture of the active agent and the CFI is achieved upon heating both components together until complete melting of said components occurs. In a preferred embodiment, the active agent and the CFI are first introduced into a solvent in which they are neither soluble nor miscible prior to being heated until complete melting of said components occurs. Preferred solvent is water. In another more preferred embodiment, complete melting of both components is achieved below 100° C. In another even more preferred embodiment, melting of both components is achieved spontaneously at room temperature, without the need for further energy.

In another embodiment of the present invention, the intimate mixture of the active agent and the CFI results, once melted, in transparent, non-solid droplets (herein after referred as “eutectic” droplets, or as “eutectic mixture”, or simply as “eutectic”) dispersed in a carrier in which the mixture is neither soluble nor miscible. It has surprisingly been found that these droplets present a substantially amorphous nature, i.e. do not present substantial crystallization. In a preferred embodiment, eutectic droplets are totally amorphous, i.e. do not contain any crystal.

In another embodiment of the present invention, the carrier can be substantially hydrophilic, for example, and can include water. In a most preferred embodiment, the carrier consists essentially of water. Thus, the pharmaceutically acceptable carrier can be substantially free of alcohol. Hence, unlike many other transdermal or topical compositions which require alcohol for effective permeation, the present eutectic compositions can be provided as substantially free of alcohol without affecting the efficacy of skin permeation of the composition. Thus, the invention provides a topical treatment without causing local skin irritation; itching or burning commonly associated with alcoholic formulations but also can successfully address skin irritations caused by alcoholic topical products. However, in another embodiment, the carrier may comprise water and a low amount of a short-chain alcohol. It has been found that addition of low amounts of a short alcohol in the composition of the present invention further stabilizes the eutectic active agent-CFI droplets. Advantageously, the presence of a low amount of a short-chain alcohol in the carrier allows for increasing the ratio of active agent to CFI able to form a stable eutectic mixture. Importantly, it is understood in any embodiment that the amount of the short-chain alcohol used as co-melting point depressant agent is too low to enable a complete solubilization of the active agent. In a preferred embodiment, the short-chain alcohol is ethanol, propanol, isopropanol, butanol, propylene glycol, polyethylene glycols, diethylene glycol mono ethyl ether, glycofurol, and mixtures thereof, and the amount of the short-chain alcohol does not exceed 50% by weight of the total composition. Preferred short-chain alcohol is ethanol, isopropanol, propylene glycol. In a more preferred embodiment, the amount of ethanol does not exceed 30% by weight of the total composition. In an even more preferred embodiment, the amount of ethanol does not exceed 15% by weight of the total composition. In a most preferred embodiment, the composition of the present invention is alcohol-free.

In another embodiment, the composition of the present invention containing the active agent as an eutectic mixture with a CFI has surprising and unexpected transdermal/or transmucosal (or topical) permeation (or penetration, respectively) enhancing properties, when compared with similar compositions not containing the said active agent as an eutectic mixture with a CFI, e.g. not containing a CFI. In a preferred embodiment, enhanced permeation properties of compositions of the present invention are witnessed by the determination of the total in vitro cumulated permeated drug amount after a defined period of time, and/or by the maximal instant drug flux profile the active agent. In a preferred embodiment, enhanced penetration properties of compositions of the present invention are witnessed by the determination of the final in vitro amount of drug in each layer of the skin after a defined period of time.

Without being held to any one theory, it is believed that the enhanced permeation is achieved by a decrease in the melting point of the active agent by the CFI, which enables a formulation of an oil-in-water emulsified system, having enhanced skin permeation or penetration properties. The enhancement mechanism for the composition of the present invention is believed to be as follows:
dQ/dt=KCvDsA/h [equation 1, from Higuchi]
wherein:
dQ/dT is the steady state rate of penetration
K is the skin vehicle partition coefficient
Cv is the concentration of the drug in the vehicle
Ds is the diffusion coefficient constant of the drug in the skin
A is the area of drug application
H is the thickness of the skin
   Furthermore,
   Jss=dQ/dt×1/A=KCvDs/h=P×ΔC [equation 2]
   with:
Jss is the transdermal flux
P is the permeability coefficient (cm.h-1) of the drug
ΔC is the concentration gradient between the donor and receptor compartments.

Thus by combining equations 1 and 2, the permeability coefficient P is proportional to the transdermal flux Jss (increase of P enables to increase Jss) or proportional to the reciprocal of the concentration gradient ΔC. Guy and Hadgraft (“Transdermal Drug Delivery”, Hadgraft and Guy, Marcel Dekker, Inc., New York and Basel, 1989, p. 72-73 have demonstrated that for numerous drugs (a) provided sink condition occur on one side of the membrane; and (b) provided an infinite dose of drug is applied to the other, then the concentration gradient ΔC is proportional to the solubility of the drug in the lipid phase of the membrane, or to the reciprocal of the melting point, as illustrated below.

[image]

Consequently, a decrease of the melting point of an active drug by forming eutectic mixtures enables an increase of concentration gradient ΔC, and also of the transdermal flux of the active ingredient as per Equation 2 above.

In another embodiment of the present invention, the presence of a CFI to transform the active agent into a eutectic mixture makes unnecessary the incorporation of further flavoring or fragrance agents to impart a pleasant odor profile to the composition of the present invention. However, it is understood that such additional flavoring or fragrance agents may be added.

According to the present invention, a transdermal or topical composition with enhanced permeation or penetration through the skin or the mucosa of at least one pharmacologically active agent is provided by providing the pharmacologically active agent in a eutectic mixture with a CFI. It is believed that forming the eutectic mixture of active agent and CFI decreases the melting point of the pharmacological active agent, thereby increasing the solubility of the pharmacologically active agent in the skin lipids and thereby enhancing skin permeation of the agent, while having recourse to very low amounts or no amounts at all of alcohol.

In one embodiment, the composition comprises a substantially alcohol-free amorphous mixture of a non-steroidal anti inflammatory drug as the active agent and of a CFI. In one preferred embodiment, the non-steroidal anti inflammatory drug is ketoprofen and the CFI is PES. In another preferred embodiment, the non-steroidal anti inflammatory drug is ibuprofen and the CFI is 4-(1,3-benzodioxol-5-yl)butan-2-one is the CFI. It has been surprisingly found that PES enhances skin permeation of the ketoprofen and that 4-(1,3-benzodioxol-5-yl)butan-2-one enhances skin permeation of the ibuprofen.

In yet another embodiment, the topical composition comprises a eutectic mixture of a local anesthetic as the active agent and a CFI. In one preferred embodiment, the non-steroidal anti inflammatory drug is lidocaine and the CFI is PES. In another preferred embodiment, it has been surprisingly found that specific ratios of lidocaine to PES can form eutectic mixtures spontaneously at ambient temperature without the need for providing further energy such as heating. Preferred ratios of lidocaine to PES are ranging from 55:45 to 45:55. Preferred ratio of lidocaine to PES is 50:50. It has been surprisingly found that PES enhances skin penetration of lidocaine within the different layers of the skin. In another preferred embodiment, the non-steroidal anti inflammatory drug is prilocalne and the CFI is PES. It has been surprisingly found that PES enhances skin penetration of prilocalne within the different layers of the skin.

In yet another embodiment, the composition comprises an amorphous mixture of an anticholinergic drug as the active agent and of a CFI. In one preferred embodiment, the anticholinergic drug is oxybutynin and the CFI is PES. In a more preferred embodiment, a low amount of ethanol may be added to further stabilize the eutectic mixtures.

In yet another embodiment, the composition comprises an amorphous mixture of a cardiovascular drug as the active agent and of a CFI. In one preferred embodiment, the cardiovascular drug is carvedilol or clonidine and the CFI is PES. In a more preferred embodiment, a low amount of ethanol may be added to further stabilize the eutectic mixtures.

In yet another embodiment, the composition comprises an amorphous mixture of an opioid analgesic drug as the active agent and of a CFI. In one preferred embodiment, the opioid analgesic drug is fentanyl and the CFI is PES. In a more preferred embodiment, a low amount of ethanol may be added to further stabilize the eutectic mixtures.

In yet another embodiment, the composition comprises an amorphous mixture of an antiparkinson drug as the active agent and of a CFI. In one preferred embodiment, the antiparkinson drug is ropinirole and the CFI is PES. In another preferred embodiment, the antiparkinson drug is pramipexole and the CFI is PES. In a more preferred embodiment, a low amount of ethanol may be added to further stabilize the eutectic mixtures.

In yet another embodiment, the composition comprises an amorphous mixture of an anti acne drug as the active agent and of a CFI. In one preferred embodiment, the anti acne drug is an antiandrogen and the CFI is PES. In a more preferred embodiment, a low amount of ethanol may be added to further stabilize the eutectic mixtures.

In yet another embodiment, the composition comprises an amorphous mixture of a sexual hormone as the active agent and of a CFI. In one preferred embodiment, the sexual hormone is testosterone and the CFI is PES. In a more preferred embodiment, a low amount of ethanol may be added to further stabilize the eutectic mixtures.

In yet another embodiment, the composition comprises an amorphous mixture of an anti-emetic drug as the active agent and of a CFI. In one preferred embodiment, the anti-emetic drug is granisetron and the CFI is PES. In a more preferred embodiment, a low amount of ethanol may be added to further stabilize the eutectic mixtures.

It is understood that it will appear obvious to the one skilled in the art that further active gents other than the ones cited herein so far may fall within the scope of the present invention. For instance, in yet another embodiment, the composition comprises a eutectic mixture of a CFI and an active agent, wherein the active agent is apomorphine, butorphanol, rivastigmine, buspirone, fentanyl, rizatriptin, tolterodine, zolmitriptan, lacidipine, tropisetron, olanzapine, methyl phenidate, testosterone, ropinirole, granisetron, nicotine, scopolamine, pramipexole, propranolol, etc . . . It has been found that the formation of a eutectic mixture with CFI in general, and PES in particular, is facilitated by using an active agent having a melting point below 250° C., more preferably below about 150° C., and even more preferably below about 100° C. or less. In this manner, the CFI and the active agent form an oily liquid mixture at ambient temperatures (typically 20° C.-25° C.). Such advantageous formation of the oily liquid mixture at ambient temperatures eliminates the requirement of any further heating steps and also facilitates handling of the mixture during subsequent manufacturing processes.

Similarly, it is understood that it will appear obvious to the one skilled in the art that other API:CFI combinations than the ones mentioned herein may exhibit similar or superior properties, by varying either the type or the concentration of the CFI. Selection of the most appropriate CFI for a given API results only from extensive experimental trials.

According to one embodiment of the invention, the transdermal or topical composition is provided as an emulsion of a discontinuous phase mixed in a continuous phase. The discontinuous phase comprises an amorphous mixture of an active agent and a CFI, preferably having a melting point below 37° C., and more preferably below 25° C. The continuous phase comprises a pharmaceutically acceptable carrier.

One or more gelling or suspension agent can be included in the pharmaceutically acceptable carrier in the present composition. Exemplary gelling agents include, but are not limited to, carbomer, carboxyethylene or polyacrylic acid such as carbomer 980 or 940 NF, 981 or 941 NF, 1382 or 1342 NF, 5984 or 934 NF, ETD 2020, 2050, 934P N, 971P N, 974P N, polycarbophils such as NOVEON AA-1, NOVEON CA1/CA2, carbomer copolymers such as PEMULEN TR1 NF or PEMULEN TR2 NF, carbomer interpolymers such as CARBOPOL ETD 2020 NF, CARBOPOL ETD 2050 NF, CARBOPOL ULTRA EZ 10, etc . . . ; cellulose derivatives such as ethylcellulose, hydroxypropylmethylcellulose (HPMC), ethyl-hydroxyethylcellulose (EHEC), carboxymethylcellulose (CMC), hydroxypropylcellulose (HPC), hydroxyethylcellulose (HEC), etc . . . ; natural gums such as arabic, xanthan, guar gums, alginates, etc . . . ; polyvinylpyrrolidone derivatives; polyoxyethylene polyoxypropylene copolymers, etc; others like chitosan, polyvinyl alcohols, pectins, veegum grades, and the like. Other suitable gelling agents to apply the present invention include, but are not limited to, carbomers. Alternatively, other gelling agents or viscosant known by those skilled in the art may also be used. The gelling agent or thickener is present from about 0.2 to about 30% w/w depending on the type of polymer, as known by one skilled in the art. A preferred concentration range of the gelling agent(s), for example, hydroxypropyl cellulose or carbomer, is a concentration of between about 0.5 and about 5 weight percent, more preferred is a concentration of between about 1 and about 3 weight percent.

One or more emulsifying agents or systems can be included in the pharmaceutically acceptable carrier in the present composition. Exemplary emulsifying agents or systems include, but are not limited to, non-ionic, cationic or anionic surfactants.

One or more additional optional ingredients can be included in the pharmaceutically acceptable carrier in the present composition depending on the desired final product. Exemplary additional optional ingredients include, but are not limited to, volatile silicones (comprising, but not limited to, hexamethyldisiloxane, octamethyltrisiloxane, decamethylcyclopentasiloxane, dimethicone, silicone elastomer blends, silicone waxes, hydrophilic silicone fluids, cyclomethicone) which are commonly used in topical compositions to impart a silky “feel” can be included; one or more buffering agent, permeation enhancers, cosolvents, antioxidants, preservatives, humectants, sequestering agents, moisturizers, emollients, colorants, fragrances, flavors, or any combination thereof.

The present topical composition is especially versatile in that it can be readily prepared in a various forms of formulations and dosage forms, including semi-solid forms with a viscosity ranging from very low (e.g., solutions, lotions) to very high (e.g., gels, creams). Thus, the present composition can be provided in any suitable form, including a gel, ointment, lotion, suspension, solution, syrup, cream, microemulsion, and aerosol spray. Further, the composition can be deposited on a patch for application on skin or a body surface, or provided as a medicated dressing. It can also be incorporated within soft gelatin liquid capsules or tablets intended to be administered by the oral route. Thus, the present invention provides an enhanced delivery of an active pharmaceutical agent in any variety of forms.

In accordance with another aspect of the invention, a process for preparing the present composition containing a eutectic mixture of a pharmacologically active agent and a CFI is provided. The process involves heating and mixing the pharmacologically active agent and the CFI within a pharmaceutically acceptable carrier to form a eutectic mixture. Preferably, the eutectic mixture has a melting point below 37° C., and more preferably below 25° C. Preferably, the eutectic mixture and the pharmaceutically acceptable carrier form an emulsion which includes a discontinuous phase including eutectic mixture and a continuous phase including the pharmaceutically acceptable carrier. Optionally, additional ingredients can be utilized depending on the desired final product. For example, an emulsifying agent or system, gelling or suspension agent, additional permeation enhancers, may be included if desired.

The present composition and method may include a dispenser for dispensing the composition for administration to a subject. The dispenser can for example be a tube or a metered-dose pump. For example and not limitation, the dispensing tube can be any of a variety of tube dispending systems provided by Alcan Packaging Cebal and other suppliers of such dispensing systems. Alternatively, the dispensing system can be any of a variety of metered-dose pump dispending systems provided by Rexam, Valois, Lablabo, and other suppliers of such dispensing systems. A variety of metered-dose dispensing systems can be utilized, such as, for example and not limitation, Twinbags dispensing system, which is manufactured by Lablabo and the Duo Omega dispensing system, which is manufactured by Airlesssystems.

Therefore, the present invention provides a simple and efficient process for producing a substantially alcohol-free transdermal or topical composition with enhanced transdermal or transmucosal properties. The use of CFI according to the present invention is simple and effective, and uniquely achieves enhanced permeation properties without requiring complex manufacturing process or additional chemical ingredients that may be irritable to the skin, while imparting a pleasant odor profile to the composition.

### EXAMPLES

The following examples are put forth so as to provide those of ordinary skill in the art with a complete disclosure and description of how to make and use the formulations, methods, and devices of the present invention, and are not intended to limit the scope of what the inventors regard as the invention. Efforts have been made to ensure accuracy with respect to numbers used (e.g., weights, temperature, volumes, etc.) but some experimental errors and deviations should be accounted for. Unless indicated otherwise, parts are parts by weight, molecular weight is weight average molecular weight, temperature is in degrees Centigrade, and pressure is at or near atmospheric.

The compositions produced according to the present invention meet the strict specifications for content and purity required of pharmaceutical products.

A. Pharmaceuticals and Reagents.

The pharmaceuticals and reagents used in the following examples can be obtained from commercial sources, for example, as follows: active drugs (e.g., oxybutynin (free-base form), from PCAS, Limay, France; ibuprofen, from Albemarle Corporation, Orangeburg, USA; ketoprofen, from Bidachem S.p.A., Fomovo s. Giovanni Italy; lidocaine (free-base form), from Hawkins Inc. Pharmaceutical Group, Minneapolis, USA; prilocalne (free-base form), from Sekhsaria Chemicals Limited, Dambivli, India; fentanyl (free-base form), from Diosynth by, Oss, Netherlands; carvedilol (free-base form), from Amino Chemicals Ltd, Malta; clonidine (free-base form), from S.I.M.S., Firenze, Italy; granisetron (free-base form) from Hangzhou Pharma & Chem, Co, Ltd, Zhejiang, China; pramipexole (free-base form) from Changzhou Huaren Chemicals, Co, Ltd, Jiangsu, China; ropinirole, from PCAS Oy, Turku, Finland; CFI (e.g., phenyl ethyl salicylate, 4-(1,3-benzodioxol-5-yl)butan-2-one, β-naphtyl isobutyl ether, ortho tertiary butyl cyclohexanol, indeno-m-dioxin tetrahydro, from IFF, New York, USA).

B. In Vitro Skin Permeation Methodology.

The in vitro human cadaver skin model has proven to be a valuable tool for the study of percutaneous absorption and the determination of topically applied drugs. The model uses human cadaver skin mounted in specially designed diffusion cells that allow the skin to be maintained at a temperature and humidity that match typical in vivo conditions (Franz, T. J., “Percutaneous absorption: on the relevance of in vitro data,” J. Invest Dermatol 64:190-195 (1975)). A finite dose (for example: 4-7 mg/cm²) of formulation is applied to the outer surface of the skin and drug absorption is measured by monitoring its rate of appearance in the receptor solution bathing the inner surface of the skin. Data defining total absorption, rate of absorption, as well as skin content can be accurately determined in this model. The method has historic precedent for accurately predicting in vivo percutaneous absorption kinetics (Franz, T. J., “The finite dose technique as a valid in vitro model for the study of percutaneous absorption in man,” In: Skin: Drug Application and Evaluation of Environmental Hazards, Current Problems in Dermatology, vol. 7, G. Simon, Z. Paster, M Klingberg, M. Kaye (Eds), Basel, Switzerland, S. Karger, pages 58-68 (1978)).

Pig skin has been found to have similar morphological and functional characteristics as human skin (Simon, G. A., et al., “The pig as an experimental animal model of percutaneous permeation in man,” Skin Pharmacol. Appl. Skin Physiol. 13(5):229-34 (2000)), as well as close permeability character to human skin (Andega, S., et al., “Comparison of the effect of fatty alcohols on the permeation of melatonin between porcine and human skin,” J. Control Release 77(1-2):17-25 (2001); Singh, S., et al., “In vitro permeability and binding of hydrocarbons in pig ear and human abdominal skin,” Drug Chem. Toxicol. 25(1):83-92 (2002); Schmook, F. P., et al., “Comparison of human skin or epidermis models with human and animal skin in in vitro percutaneous absorption,” Int. J. Pharm. 215(1-2):51-6 (2001)). Accordingly, pig skin may be used for preliminary development studies and human skin used for final permeation studies. Pig skin can be prepared essentially as described below for human skin.

(i) Skin Preparation.

Percutaneous absorption was measured using the in vitro cadaver skin finite dose technique. Cryo-preserved, human cadaver trunk skin was obtained from a skin bank and stored in water-impermeable plastic bags at <−70° C. until used.

Prior to the experiment, skin was removed from the bag, placed in approximately 37° C. water for five minutes, and then cut into sections large enough to fit on 1 cm² Franz Cells (Crown Glass Co., Somerville, N.J.). Briefly, skin samples were prepared as follows. A small volume of phosphate buffered saline (PBS) was used to cover the bottom of the Petri dishes. Skin disks generally depleted of fat layers were placed in the Petri dishes for hydration. A Stadie-Riggs manual tissue microtome was used for slicing excised skin samples. Approximately 2 mL of PBS was placed into the middle cavity of the microtome as slicing lubricant. Skin disks were placed, dermal side up, into the middle cavity of the microtome. Filter paper was soaked with PBS, inserted in the cavity just above the skin disk. The filter paper prevented the dermis from sliding onto the top of the cutting block and helped to insure more precise cutting. When all three blades of the microtome were assembled, the microtome was turned into the upright position. Using a regular and careful sawing motion the skin tissue was sliced in cross-section. The skin tissue slice was removed with the tweezers and placed in the Petri dish for hydration. Each skin slice was wrapped in PARAFILM (Pechiney Plastic Packaging, Inc., Chicago, II) laboratory film and placed in water-impermeable plastic bags. Skin samples were identified by the donor and the provider code. If further storage was necessary, the skin slices were stored in the freezer at −20° C. until further use.

The epidermal cell (chimney) was left open to ambient laboratory conditions. The dermal cell was filled with receptor solution. Receptor solution for in vitro skin permeations was typically an isotonic saline at physiological pH. The receptor solution may also contain a drug solubilizer, for example, to increase lipophilic drug solubility in the receptor phase. The receptor solution was typically a phosphate buffered saline at approximately pH 7.4 (PBS, pH 7.4; European Pharmacopeia, 3rd Edition, Suppl. 1999, p. 192, No. 4005000) with addition of 2% Volpo N20 (oleyl ether of polyethylene glycol—a nonionic surfactant with HLB 15.5 obtained by ethoxylation (20 moles) of oleyl alcohol (C18:1)). This solubilizer is currently used for in vitro skin permeations and is known not to affect skin permeability (Bronaugh R. L., “Determination of percutaneous absorption by in vitro techniques,” in: Bronaugh R. L., Maibach H. I. (Eds.), “Percutaneous absorption,” Dekker, New York (1985); Brain K. R., Walters K. A., Watkinson A. C., Investigation of skin permeation in vitro, in: Roberts M. S., Walters K. A. (Eds.), Dermal absorption and toxicity assessment, Dekker, New York (1998)).

All cells were mounted in a diffusion apparatus in which the dermal bathing solution (i.e., the receptor solution) was stirred magnetically at approximately 600 RPM and skin surface temperature maintained at 33.0°±1.0° C.

Integrity of each skin section was determined before application of the test products by measurement of trans epidermal water loss (TEWL), using a TM 210 Tewameter (Courage-Khazaka, Germany). Differences between skin sections was determined statistically using unpaired p-test.

(ii) Dosing and Sample Collection.

(a) Franz Cell.

Just prior to dosing with the formulations described herein, the chimney was removed from the Franz Cell to allow full access to the epidermal surface of the skin. The formulations were typically applied to the skin section using a positive displacement pipette set to deliver approximately 6.25 uL (6.25 uL/1 cm²). The dose was spread throughout the surface with the TEFLON® (E.I. Du Pont De Nemours And Company Corporation, Wilmington Del.) tip of the pipette. Five to ten minutes after application the chimney portion of the Franz Cell was replaced. Experiments were performed under non-occlusive conditions. Spare cells were not dosed, but sampled, to evaluate for interfering substances during the analytical analysis.

At pre-selected time intervals after test formulation application (e.g., 2, 4, 8, 12, 16, and 24 h) the receptor solution was removed in its entirety replaced with fresh solution (0.1× Phosphate Buffered Saline with Volpo (Croda, Inc., Parsippany, N.J.), and an aliquot taken for analysis. Prior to administration of the topical test formulations to the skin section, the receptor solution was replaced with a fresh solution of Volpo-PBS. (Volpo (Oleth-20) is a non-ionic surfactant known to increase the aqueous solubility of poorly water-soluble compounds. Volpo in the receptor solution insured diffusion sink conditions during percutaneous absorption, and is known not to affect the barrier properties of the test skin.)

Skin samples from three cadaver skin donors were prepared and mounted onto cells. Typically, each formulation was tested in 4 replicates (3 different donors).

Each formulation was applied, typically, to triplicate sections for each donor. The receptor solution samples were typically collected at 2, 4, 8, 12, 16, and 24 hours after dosing. The receptor solution used was 1:10 PBS+0.1% Volpo. Differences between formulations were evaluated for statistical differences using standard statistical analysis, for example, the Student's t-Test.

After the last sample was collected, the surface was washed twice (0.5 mL volumes) with 50:50 ethanol:water twice to collect un-absorbed formulation from the surface of the skin. Following the wash, the skin was removed from the chamber, split into epidermis and dermis, and each extracted overnight in 50:50 ethanol:water for 24 hours prior to further analysis.

(b) Automatic Sampling

Automatic sampling was carried out essentially as described under “(a) Franz cell” above, with the exception that multiple cells were used coupled with an automatic sampling system. Skin from a single donor was cut into multiple smaller sections (e.g., punched skin disks cut to approximately 34 mm diameter) large enough to fit on 1.0 cm² Franz diffusion cells (Crown Glass Co., Somerville, N.J.). Skin thickness was typically between 330 and 700 um, with a mean of 523 um (+19.5%).

Each dermal chamber was filled to capacity with a receptor solution (e.g., phosphate-buffered isotonic saline (PBS), pH 7.4±0.1, plus 2% Volpo), and the epidermal chamber was left open to ambient laboratory environment. The cells were then placed in a diffusion apparatus in which the dermal receptor solution was stirred magnetically at ˜600 RPM and its temperature maintained to achieve a skin surface temperature of 32.0±1.0° C.

Typically, a single formulation was dosed to 2-3 chambers (comprising the same donor skin) at a target dose of about 5 uL/1.0 cm² using a calibrated positive displacement pipette. At pre-selected times after dosing, (e.g., 2, 4, 8, 12, 16, and 24 h) the receptor solution was sampled and a predetermined volume aliquot saved for subsequent analysis. Sampling was performed using a Microette autosampler (Hanson Research, Chatsworth, Calif.).

Following the last receptor solution sample, the surface was washed and the skin collected for analysis as described herein.

(iii) Analytical Quantification Methods.

Quantification of active agents was by High Performance Liquid Chromatography (HPLC) with Diode-Array and Mass spectrometry detector (HPLC/MS). Briefly, HPLC was conducted on a HEWLETT-PACKARD® (Hewlett-Packard Company, Palo Alto, Calif.) 1100 Series system with diode-array UV detector with MS detector. Appropriate solvent systems were run through appropriate columns at an appropriate flow rate. Samples were injected. Peak areas were quantified to concentration using an external standard curve prepared from the neat standard.

(iv) Data Analysis.

The permeation studies and the biodistribution studies (or mass balance studies) described herein provide data to obtain different profiles of the transdermal absorption of drugs through the skin as a function of time.

The absolute kinetic profile shows the mean cumulated drug permeated amount (e.g., μg/cm²) as a function of time (e.g., hours) and thus provides an evaluation of the daily absorbed dose (amount of drug transdermally absorbed after 24 hours of permeation).

The relative kinetic profile shows the mean cumulated drug permeated amount (e.g., percent) as a function of time (e.g., hours) and thus allows an evaluation of the percentage of the applied drug that is transdermally absorbed after a given time.

The flux profile shows the mean drug instant flux [e.g., μg/cm²/h] as a function of time (e.g., hours) and provides a time the steady-state flux is reached. This profile also provides an evaluation of the value of this steady-state flux. This value corresponds to the mean flux obtained at steady-state.

The mass balance profile shows distribution of the active compound (e.g., percent) within the different compartments as a function of time (e.g., hours), and more particularly within the stratum corneum, the epidermis, the dermis, the receptor compartment.

These different profiles provide means to evaluate, characterize, and compare formulations, as well as to assess the pharmaceutical efficacy of formulations and consequently, to optimize prototype formulations.

C. Formulation of Pharmaceutical Compositions.

Following here is an exemplary description of the manufacturing process used to make the pharmaceutical compositions of the present invention. Generally, the active agent and the CFI were weighed separately and added to the carrier, e.g. water alone or water and ethanol, in which neither the active agent nor the CFI do entirely solubilize. The herein obtained drug suspension was then heated until complete melting of the components, witnesses by the formation of clear, transparent oily droplets, and let cooled down. If desired, further ingredients such as cosolvents, buffering agents, antioxidants, preservatives, permeation enhancers, etc . . . were added under mechanical stirring. Emulsifying and/or thickening agents were ultimately incorporated under stirring to yield a homogeneous emulsified semi-solid dosage form. If desired, the pH was then adjusted to a specified pH, and water added quantum sufficiat (q.s.).

As used herein, some of the terms are abbreviated as follows:
API Active Pharmaceutical Ingredient
CFI Chemical Fragrance Ingredient
PES Phenyl ethyl salicylate
BDB (4-(1,3-benzodioxol-5-yl)butan-2-one
NIE β-naphtyl isobutyl ether
TID tetrahydro indeno-m-dioxin
TBC Ortho tertiary butyl cyclohexanol

### Example 1

### Melting Point Depression Effects of CFI on Local Anaesthetics

Melting point depression effects of CFI on local anaesthetic (LA) drugs were investigated.

### Example 1.1

### Dry Blending of Bulk Powdered CFI and Local Anaesthetics

Various mixtures of local anaesthetic drugs and CFI, with the ratio of LA to CFI ranging from 90:10 to 10:90, were prepared in HPLC glass vials. Vials were then sealed, placed in a water bath, and then heated until complete melting of powders occurred in all vials. Vials were then allowed to cool down to the ambient laboratory temperature (typically about 21°-25° C.) and maintained at this temperature for at least 24 hours. The samples were then checked visually. Surprisingly, some mixtures of LA and CFI were maintained as stable transparent droplets.

Table 1 below lists some ingredients studied and some melting point depression effects achieved for each LA with LA-CFI eutectic mixtures.

TABLE 1
Melting Point Depression Effects of CFI on Local Anaesthetics

| | LA | LA:PES |
|---|---|---|
| LA | melting point | mixtures |
| Benzocaine | 92° C. | Liquid oil obtained at LA:NIE ratio of |
| | | 10:90 at room temperature |
| | | Liquid oil obtained at LA:PES ratio of |
| | | 10:90 at room temperature |
| | | Liquid oil obtained at LA:BDB ratio of |
| | | 20:80 at room temperature |
| Lidocaine | 68° C.-69° C. | Liquid oil obtained at LA:PES ratios of |
| | | 70:30 and below at room temperature |
| Prilocaine | 37° C.-38° C. | Liquid oil obtained at LA:PES ratios of |
| | | 80:20 to 50:50 at room temperature |

(ratios are expressed as NSAID:CFI ratios)

Preferred LA to be used with the herein disclosed invention have a melting point lower than 200° C. More preferred LA have a melting point lower than 150° C. Even more preferred LA have a melting point lower than 115° C.

Differential scanning calorimetry (DSC) thermal analyses on lidocaine:PES mixtures were conducted with a DSC-7 Perkin Elmer (St. Quentin en Yvelines, France) using aluminum pans of 50 μl (pan, part No. B014-3021 and cover, part No. B014-3004) hermetically sealed. The reference was an empty, hermetically sealed aluminum pan. The calibration of the calorimeter was made with lauric acid (m.p. 43.7.C, ΔHm=8.53 kcal/mol). The calorimeter was operated in a glove box under a stream of dried air. Data was analyzed using PYRIS™ software.

The transitions existing for both pure compounds were determined on a wide range of temperature (−50≦T≦+150° C.).

The possible reversibility of the transition was determined by performing temperature cycling, i.e. one heating cycle (heating rate: 5° C./min) from −50° C. to +150° C., then one cooling cycle (cooling rate: 10° C./min) from +150° C. to −50° C., and then one heating cycle again (heating rate: 5° C./min, from −50° C. to +150° C.).

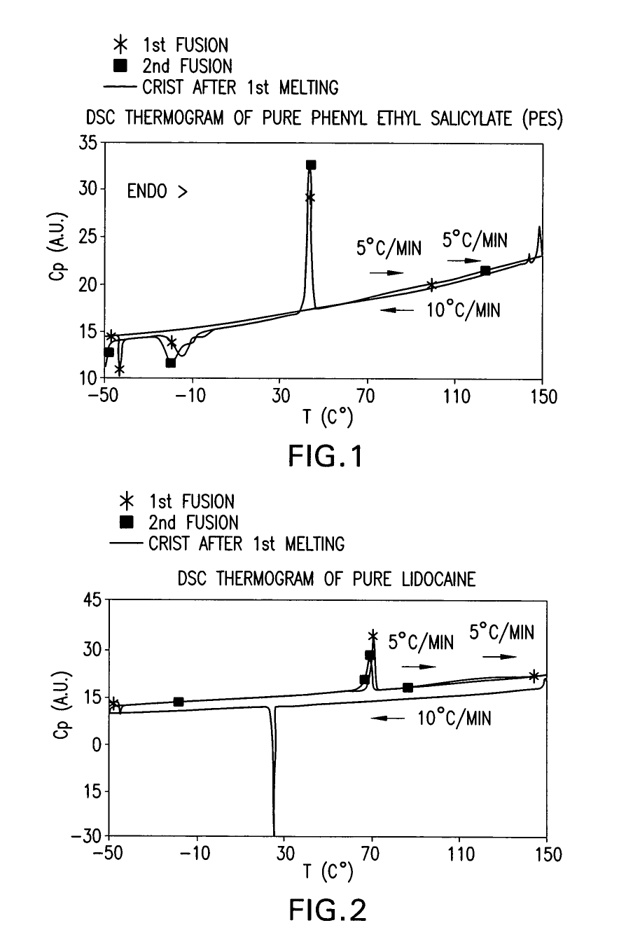

The DSC thermogram of pure PES is presented in FIG. 1. In FIG. 1, the vertical axis is Apparent Specific Heat (C.p), expressed in arbitrary units (A.U.); the horizontal axis is Temperature (in degree Celsius ° C.). The data points for first fusion cycle are presented as crosses, the data points for second fusion cycle are presented as squares, and the data points for cooling cycle are presented as plain line. Powdered PES sample showed a sharp melting peak at about 43° C. during the first DSC heating cycle at 5° C./min. The subsequent cooling cycle presented no visible thermal event, hence demonstrating the irreversibility of the melting of PES, or, at least, that PES presents a significant delay in crystallization. The latter hypothesis was confirmed by the second heating cycle, which showed an important and broad exothermic peak at about −20° C. interpreted as a difficult re-crystallization of the sample. The resulting crystalline form ultimately melted at about 40° C.

The DSC thermogram of pure lidocaine (LID) is presented in FIG. 2. In FIG. 2, the vertical axis is Apparent Specific Heat (C.p), expressed in arbitrary units (A.U.); the horizontal axis is Temperature (in degree Celsius ° C.). The data points for first fusion cycle are presented as crosses, the data points for second fusion cycle are presented as squares, and the data points for cooling cycle are presented as plain line. Powdered LID sample showed a sharp melting peak at about 68-69° C. during the first DSC heating cycle. The subsequent cooling cycle showed a sharp exothermic peak at about 25° C., corresponding to crystallization. This delay in the crystallization was interpreted as a very important supercooling of the order of about 45° C. Second heating cycle presented again the melting peak at about 66-67° C.

DSC thermal analyses were then carried out on LID:PES mixtures, using same experimental conditions as described herein before, except for the scan rates that were reduced to as 2 and 3° C./min in an attempt to have more accurate description of phenomenon occurring around melting/crystallization temperatures. Range of temperature was narrowed to from −50° C. to +70°/80° C. only.

LID to PES ratios were then investigated. To prevent sample-holder leakage, powder were directly placed and weighted in the DSC capsules. First heating will ensure the mixing once compounds are melted. So the first recording is informative regarding the mixing of components and the possible formation of a liquid by simple powder mixing (see below).

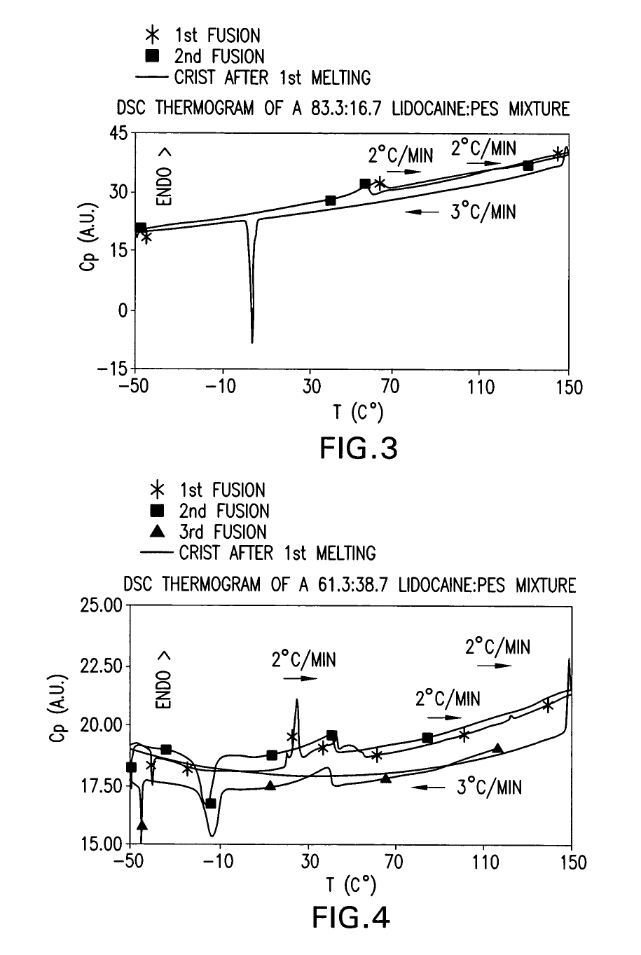

A LID:PES 83.3:16.7 mixture was prepared and analyzed for DSC. See FIG. 3, wherein the vertical axis is Apparent Specific Heat (C.p), expressed in arbitrary units (A.U.); the horizontal axis is Temperature (in degree Celsius ° C.); and wherein the data points for first fusion cycle are presented as crosses, the data points for second fusion cycle are presented as squares, and the data points for cooling cycle are presented as plain line. The mixture mainly displays behavior of pure LID. However, the exotherm corresponding to crystallization of the mixture is different than the one of the pure LID: broadening of the crystallization peak base is outstanding. The specific heat jump which is observed at about −10° C. represents the beginning of the broad melting peak ending at about 60° C. on second heating. The important supercooling observed for pure LID is further shifted by about 25° C. toward lower temperatures.

A LID:PES 61.3:38.7 mixture was prepared and analyzed for DSC. See FIG. 4, wherein the vertical axis is Apparent Specific Heat (C.p), expressed in arbitrary units (A.U.); the horizontal axis is Temperature (in degree Celsius ° C.); and wherein the data points for first fusion cycle are presented as crosses, the data points for second fusion cycle are presented as squares, the data points for cooling cycle subsequent to first heating cycle are presented as plain line, and the data points for third heating cycle are presented as upright triangles (a fourth heating recording was carried out, but is not shown on FIG. 1C, since it was totally superimposed to the third one). The mixture mainly displays behavior of pure LID. First heating showed that the two compounds i) are not yet mixed, or ii) are not yet melted. Once again, no crystallization exotherm was recorded upon cooling down, similarly to what was observed with pure PES, but conversely to what was observed with previous LID:PES 83.3:16.7 mixture. A specific heat jump is observed once at −10° C. during second heating, and a broad exotherm ending at about 37° C. is observed. It can be concluded that this LID:PES 61.3:38.7 mixture, wherein LID is the main compound, still mainly displayed behavior of pure LID, as observed previously. Noteworthy however, the important supercooling observed for pure LID and for previous mixture is even further shifted by more than 50° C. toward lower temperatures.

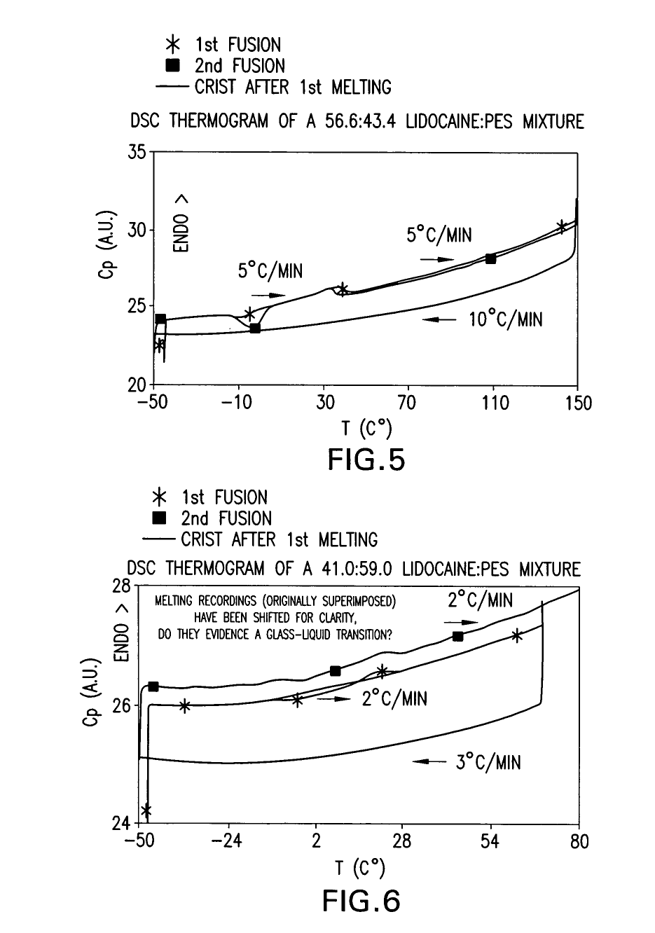

A LID:PES 56.6:43.4 mixture was prepared and analyzed for DSC. See FIG. 5, wherein the vertical axis is Apparent Specific Heat (C.p), expressed in arbitrary units (A.U.); the horizontal axis is Temperature (in degree Celsius ° C.); and wherein the data points for first fusion cycle are presented as crosses, the data points for second fusion cycle are presented as squares, and the data points for cooling cycle are presented as plain line. Surprisingly, this mixture (as well as mixtures with about one part of LID for about one part of PES) spontaneously leads to the formation of a liquid phase by simple mixing the powders together. The formation of a liquid phase by simple mixing of powders can be attributed to either an exothermic process or the formation of a eutectic formation, if not both. After a first screening of possible chemical reactions between both compounds the former hypothesis was ruled out, since no evidence of possible chemical reaction was found. First heating cycle showed a very broad exothermic peak starting from about −10° C. and ending at about +35° C. While again, no crystallization exotherm is recorded on cooling as observed for pure PES and previous LID:PES 61.3:38.7 mixture, one crystallization peak is observed on second heating (from about −10° C. to about 0° C.). Melting point of this mixed crystalline form is further decreased by about 5° C. No specific heat jump has been observed.

Concentration of PES in the mixture was further increased so that the PES became the main compound. A LID:PES 41.0:59.0 mixture was prepared and analyzed for DSC. See FIG. 6, wherein the vertical axis is Apparent Specific Heat (C.p), expressed in arbitrary units (A.U.); the horizontal axis is Temperature (in degree Celsius ° C.); and wherein the data points for first fusion cycle are presented as crosses, the data points for second fusion cycle are presented as squares, and the data points for cooling cycle are presented as plain line. Final melting point of the mixture is even further decreased again by about 8° C. (second heating cycle). No specific heat jump corresponding to glass transition has been observed.

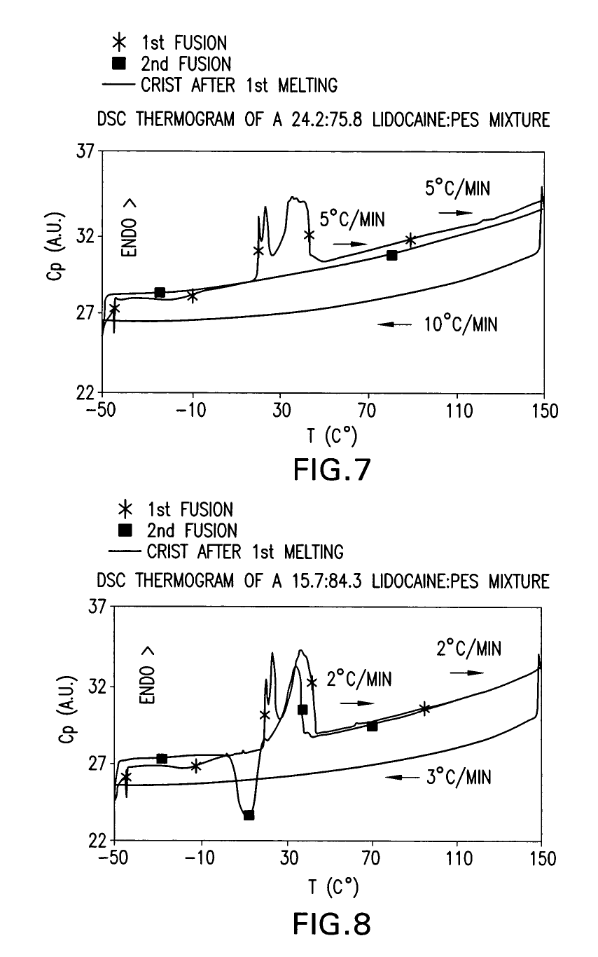

Concentration of PES in the mixture was further increased. A LID:PES 24.2:75.8 mixture was prepared and analyzed for DSC. See FIG. 7, wherein the vertical axis is Apparent Specific Heat (C.p), expressed in arbitrary units (A.U.); the horizontal axis is Temperature (in degree Celsius ° C.); and wherein the data points for first fusion cycle are presented as crosses, the data points for second fusion cycle are presented as squares, and the data points for cooling cycle are presented as plain line. Final melting point of the mixture is even further decreased again by about 8° C. (second heating cycle). No melting point is observed during second heating.

Concentration of PES in the mixture was even further increased. A LID:PES 15.7:84.3 mixture was prepared and analyzed for DSC. See FIG. 8, wherein the vertical axis is Apparent Specific Heat (C.p), expressed in arbitrary units (A.U.); the horizontal axis is Temperature (in degree Celsius ° C.); and wherein the data points for first fusion cycle are presented as crosses, the data points for second fusion cycle are presented as squares, and the data points for cooling cycle are presented as plain line. Final melting point of the mixture is increased by about 12° C. showing that mixture behavior of this mixture is clearly dominated by PES. No specific heat jump corresponding to glass transition has been observed. A small peak develops at about 20° C.

All the examples aforementioned demonstrate that addition of PES to LID does alter the thermal events (melting and crystallization) of LID. More particularly, addition of PES does enhance supercooling (or delay in crystallization) observed for LID, by forming a new intimate mixture which displays lowered melting point. For illustration, a 1:1 mixture is liquid at room temperature, i.e. has a melting point of about 20-25° C. at maximum.

All the examples aforementioned demonstrate that lowering of melting point of LID is not PES concentration dependent, i.e. the more PES the lower the melting point of the mixture containing LID is: see, e.g., LID:PES 15.7:84.3 mixture.

Therefore all the examples aforementioned demonstrate the benefit of adding specific amount of PES to an active drug in order to decrease melting point of said active drug.

Dry blends of LID with other CFI were prepared, at a ratio close to 50:50. Surprisingly, as observed beforehand with PES, LID:NIE, LID:TBC and LID:TID mixtures spontaneously melt at room temperature (about 23° C.) when simply weighed altogether in a glass weighing dish, conversely to LID:BDB mixture.

### Example 1.2

### Aqueous Blending of Bulk Powdered CFI and Local Anaesthetics

Various mixtures of local anaesthetic drugs and CFI, with the ratio of API to CFI ranging from 90:10 to 10:90, were prepared in glass vials. Vials were then filled with a known amount of water, sealed, placed in a water bath, and then heated until complete melting of suspended powders occurred in all vials. Vials were then allowed to cool down to the ambient laboratory temperature (typically about 21° C.) and maintained at this temperature. The samples were then checked visually. Surprisingly, some mixtures of LA and CFI were maintained as stable transparent droplets. For instance, a composition containing lidocaine 2.50% w/w and PES 2.50% w/w in water was prepared as described herein above. Its aspect was visually maintained even after more than a 13 month-storage period at ambient temperature in the dark. More particularly, droplets were still transparent and substantially colorless. Droplets were easily re-suspended by gentle hand-shaking. A small aliquot of the re-constituted emulsion showed no crystallization when checked microscopically. Similarly, stable droplets of oily amorphous lidocaine can also be achieved with 4-(1,3-benzodioxol-5-yl)butan-2-one, β-naphtyl isobutyl ether, indeno-m-dioxin tetrahydro, and ortho tertiary butyl cyclohexanol. Some differences are noticeable however: mixtures of LID with PES and 4-(1,3-benzodioxol-5-yl)butan-2-one are prone to form a very few, large droplets by coalescence (aggregation), although lidocaine mixtures with β-naphtyl isobutyl ether and indeno-m-dioxin tetrahydro tend to form numerous small vesicles; oil droplets formed by lidocaine mixtures with ortho tertiary butyl cyclohexanol are so small that they do not sediment (conversely to all the previously cited mixtures) and make the composition almost transparent, as a microemulsion. Since the various CFI exhibit different chemical and physical properties, such as octanol-water partition coefficient (i.e. degree of lipophilicity), it is hypothesized that selection of the CFI enabling formation of amorphous oily lidocaine has an impact on the final lipophily of the API:CFI mixture, and consequently may affect the diffusion of said mixture within the different skin layers.

For illustration, lidocaine 1% w/w can be solubilized in a hydro-alcoholic mixture containing at least 20% by weight of the total mixture of ethanol. Similarly, solubilization of lidocaine 2.50% w/w would require about a hydro-alcoholic mixture containing at least 35-40% by weight of the total mixture of ethanol. However, lidocaine would crystallize massively upon—quick—evaporation of ethanol, hence impairing lidocaine skin permeation.

In view of the foregoing, it is demonstrated that alcohol-free semi-solid formulations of lidocaine wherein lidocaine is present under an amorphous, liquid state can easily be achieved by the use of the present invention, i.e. by decreasing melting point of lidocaine thanks to a chemical fragrance ingredient.

### Example 1.3

### Semi-Solid Gel Composition of Amorphous, Non-Solid LA

Droplets of LA:CFI obtained by practice of the present invention can be finely divided by gentle mixing and then suspended homogeneously thanks to the use of emulsifiers and/or gelling agents known to the one skilled in the art of compounding pharmaceutical products.

Following exemplary formulations comprise should not be interpreted as limitative, and variations may appear obvious to the man in the art.

### Example 1.3.1

### Lidocaine Emulgel

2.50 g of lidocaine base and 2.50 g of phenyl ethyl salicylate are added to 20.0 g of purified water into a sealed container and heated in a water-bath until formation of transparent droplets, and then let cooled down to room temperature. 5.0 g of PEG-8 caprylic/capric triglycerides (LABRASOL®, Gattefosse, Saint Priest, France) are then added under gentle mixing to the lidocaine emulsion. Separately, 1.00 g carbomer (Carbopol 974P for instance) is dispersed under gentle mixing in about 60.0 g of purified water until a lump-free homogeneous dispersion is obtained. The lidocaine-phenyl ethyl salicylate emulsion is then added to the carbomer dispersion and homogenized under stirring. Triethanolamine solution (0.4 g of triethanolamine dissolved in about 8.6 g of purified water) is then added to the active carbomer dispersion under vigorous stirring. A white, homogeneous, opalescent creamy gel is then formed upon neutralization. Microscopic examination (STEMI 2000C microscope, Carl Zeiss, Germany) reveals absence of drug crystals.

The obtained alcohol-free gel presents a pleasant balsamic, floral, rose fragrance note.

### Example 1.3.2

### Prilocalne Emulgel

Same as Example 1.3.1 except that lidocaine is replaced by prilocalne.

### Example 1.3.3

### Lidocaine Silicone Gel

2.50 g of lidocaine base and 2.50 g of phenyl ethyl salicylate are weighed in a glass vial, which is then sealed and placed in a water bath. Lidocaine and phenyl ethyl salicylate powdered mixture is then heated until complete melting. The obtained transparent oil is then let cooled down to room temperature. The lidocaine oil is gently dispersed in 23.8 g of cyclomethicone 5NF (Dow Corning Corporation, Midland, USA). 71.2 g of silicon elastomer ST 10 (Dow Corning Corporation, Midland, USA) are then slowly incorporated under mixing into the fluid silicon emulsion obtained beforehand. A firm homogeneous, practically transparent gel is obtained, with a pleasant silky touch and floral fragrance note.

### Example 1.3.4

### Lidocaine Silicone Aqueous Gel

2.50 g of lidocaine base and 2.50 g of phenyl ethyl salicylate are weighed in a glass vial, which is then sealed and placed in a water bath. Lidocaine and phenyl ethyl salicylate powdered mixture is then heated until complete melting. The obtained transparent oil is then let cooled down to room temperature. Separately, 3.0 g of SIMULGEL PHA 600 (Seppic, Paris, France) is mixed with 11.5 g of 34.5 g of cyclomethicone 5NF and 34.5 g of silicon elastomer ST 10. The lidocaine-phenyl ethyl salicylate emulsion is then added to the silicon phase and homogenized under stirring. A smooth, homogeneous, whitish, light gel is obtained, with a pleasant silky touch and floral fragrance note.

### Example 1.3.5

### Prilocalne Silicone Aqueous Gel

Same as Example 1.3.4 except that lidocaine is replaced by prilocalne.

### Example 1.3.6

### Lidocaine Emulgel

Same as Example 1.3.1 except that phenyl ethyl salicylate is replaced by 4-(1,3-benzodioxol-5-yl)butan-2-one.

### Example 1.3.7

### Lidocaine Emulgel

Same as Example 1.3.1 except that phenyl ethyl salicylate is replaced by β-naphtyl isobutyl ether.

### Example 1.3.8

### Lidocaine Emulgel

Same as Example 1.3.1 except that phenyl ethyl salicylate is replaced by indeno-m-dioxin tetrahydro.

### Example 1.3.9

### Lidocaine Emulgel

Same as Example 1.3.1 except that phenyl ethyl salicylate is replaced by ortho tertiary butyl cyclohexanol.

### Example 1.3.10

### Lidocaine Emulgel

Same as Example 1.3.1 except that phenyl ethyl salicylate is partially replaced by ortho tertiary butyl cyclohexanol.

### Example 1.4

### In Vitro Skin Penetration Studies

Formulations disclosed in Examples 1.3.5 was compared to a marketed reference drug product, EMLA®, which consists of a eutectic mixture of lidocaine and prilocalne dispersed in a water-based gel matrix. Composition of EMLA is as follows: 2.5% lidocaine, 2.5% prilocalne, 1.0% Carbopol 934P, 1.9% polyoxyethylene hydrogenated castor oil, 1 M sodium hydroxide qs pH 8.7-9.7 (between 10.0% and 15.0%, based on experimental trials), purified water qs.

Table 2 herein after presents exemplary components of lidocaine-prilocalne gel formulations used in the following experiments.

TABLE 2
Composition of Formulations (% w/w)
FORMULATION

| | Denomination | | |
|---|---|---|---|
| | | “EMLA ® | Example |
| | EMLA ® | Silicone” | 1.3.5 |
| | pH | | |
| | 9.5 | 9.3 | 8.8 |
| Composition | % w/w | % w/w | % w/w |
| Lidocaine base | 2.50 | 2.50 | 2.50 |
| Prilocaine base | 2.50 | 2.50 | — |
| Phenyl ethyl salicylate | — | — | 2.50 |
| Carbomer (Carbopol C934P) | 1.00 | — | — |
| Polyoxyethylene | 1.90 | — | — |
| hydrogenated castor oil | | | |
| Sodium hydroxide, 1M | qs pH 8.7-9.7 | — | — |
| Water | qs | 46.00 | 46.00 |
| ST Cyclomethicone 5 NF | — | 11.50 | 11.50 |
| ST Elastomer 10 | — | 34.50 | 34.50 |
| Simulgel PHA 600 | — | 3.00 | 3.00 |
| Total | 100.00 | 100.00 | 100.00 |

Fresh sliced pig ear skin was used for the permeation studies using Franz cells as described in section “B—In Vitro Skin Permeation Methodology”.

Skin biodistribution of lidocaine and prilocalne using formulations exemplified in Table 2 herein above was evaluated using an apparatus for automated sampling (described in the Materials and Methods Section). Individual gel amounts applied to tested skin samples were approximately 50 mg. Studies were performed according to OECD (Organization for Economic Cooperation and Development) guidelines (Organization for Economic Co-operation and Development (OECD), Environment Directorate. “Guidance document for the conduct of skin absorption studies,” OECD series on testing and assessment, No. 28. Paris, version 5 Mar. 2004). The results presented in Table 3 show the mean values of recovered amount of lidocaine in each skin compartment 4 hours after skin application of the formulations. Results are expressed in percent of applied lidocaine dose.

TABLE 3
Lidocaine Recovery in Skin Compartments After 4 hours Skin Contact

| | Epidermal | Dermal | Skin |
|---|---|---|---|
| | absorption. | absorption | absorption |
| Formulation | [SC + E] | [D] | [SC + E + D] |
| EMLA ® | 6.9 | 2.7 | 9.6 |
| “EMLA ® Silicone” | 5.2 | 2.9 | 8.0 |
| Example 1.3.5 | 4.5 | 5.2 | 9.7 |

SC Stratum Corneum
E Epidermis
D Dermis

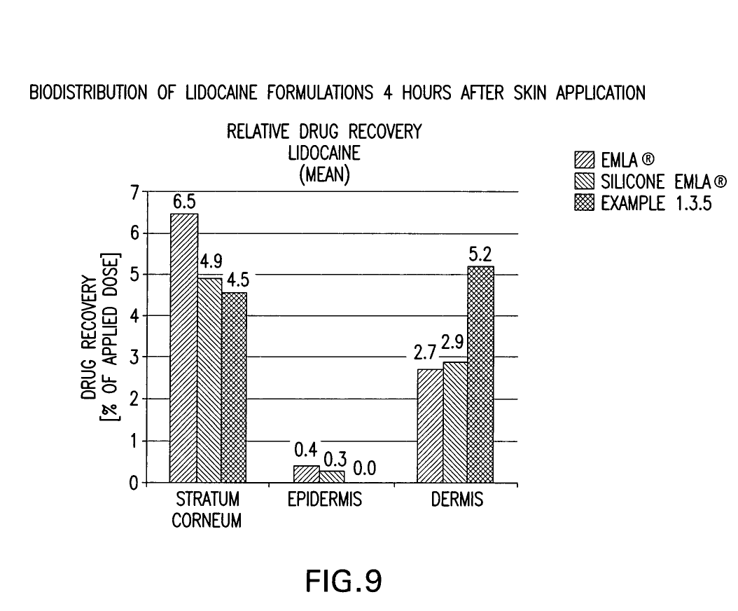

The relative drug recovery profile of lidocaine 4 hours after topical application of the formulations exemplified in Table 2 herein before is presented in FIG. 9. In FIG. 9, the vertical axis is Drug Recovery (expressed as percent of applied dose), the horizontal axis is Skin Compartment. The data points for EMLA® are presented as white dot, the data points for “EMLA® Silicone” are presented as wide downward diagonal, and the data points for Example 1.3.5 are presented as plaid.

Comparing EMLA® and “EMLA® Silicone” gives information on effect of vehicle, i.e. a water-based vehicle in EMLA® versus a silicon-water vehicle in “EMLA® Silicone”, lidocaine being present in both formulations as a eutectic formed with prilocalne in a 1:1 ratio. Data show similar drug recovery in the dermal layer (2.7% versus 2.9%, respectively). Lidocaine absorption in outermost layers of the skin (stratum corneum and epidermis) is slightly higher in EMLA® than in “EMLA® Silicone” (6.9% versus 5.2%, respectively).

Comparing “EMLA® Silicone” and Example 1.3.5 gives information on effect of compound used to form the 1:1 eutectic mixture of lidocaine, i.e. prilocalne in “EMLA® Silicone” and PES in Example 1.3.5, the drug vehicle being the same silicon-water vehicle in both formulations. Data show a dermal recovery of lidocaine significantly higher in Example 1.3.5 than in “EMLA® Silicone” (80% improvement, p=0.05), suggesting therefore a better dermal absorption.

Comparing EMLA® and Example 1.3.5 show similar total skin absorption (9.6% versus 9.7%, respectively). However, this similarity hides significant important discrepancies, since the amount of lidocaine in the dermis accounts for only about 28% of total skin absorption in EMLA®, while it is about twice more (about 54%) in Example 1.3.5.

This data demonstrate the greater ability of the formulation of the present invention to target the dermis when delivering lidocaine. Noteworthy, nerve endings, which represent the site of action of local anaesthetics such as lidocaine, are mainly located in the dermis.

Therefore, this data demonstrate the greater potential of the formulation of the present invention for topical application of lidocaine in particular and more generally of local anaesthetics.

### Example 2

### Melting Point Depression Effects of CFI on Non Steroidal Anti Inflammatory Drugs

Melting point depression effects of CFI on non steroidal anti inflammatory drugs (NSAID) drugs were investigated.

### Example 2.1

### Dry Blending of Bulk Powdered CFI and NSAID

Various mixtures of NSAID and CFI, with the ratio of NSAID to CFI ranging from 90:10 to 10:90, were prepared as described in Example 1.1. The samples were then checked visually. Surprisingly, some mixtures of NSAID and CFI were maintained as stable transparent droplets.

Table 4 below lists some ingredients studied and some melting point depression effects achieved for each NSAID with NSAID-CFI eutectic mixtures.

TABLE 4
Melting Point Depression Effects of CFI on NSAID

| | NSAID | LA:PES |
|---|---|---|
| NSAID | melting point | mixtures |
| Ibuprofen | 75° C.-77° C. | Liquid oil with PES at 90:10 to |
| | | 50:50 at room temperature |
| | | Liquid oil with BDB from 60:40 to about |
| | | 40:60 at room temperature |
| | | Liquid oil with NIE at 70:30 room |
| | | temperature |
| Ketoprofen | 68° C.-69° C. | Liquid oil obtained at API:PES ratios |
| | | of 80:20 and 70:30 at room temperature |
| | | Liquid oil obtained at API:TID ratios of |
| | | about 50:50 and below at room |
| | | temperature |

(ratios are expressed as NSAID:CFI ratios)

Differential scanning calorimetry (DSC) thermal analyses on NSAID:CFI mixtures were conducted as described in Example 1.1 herein above.

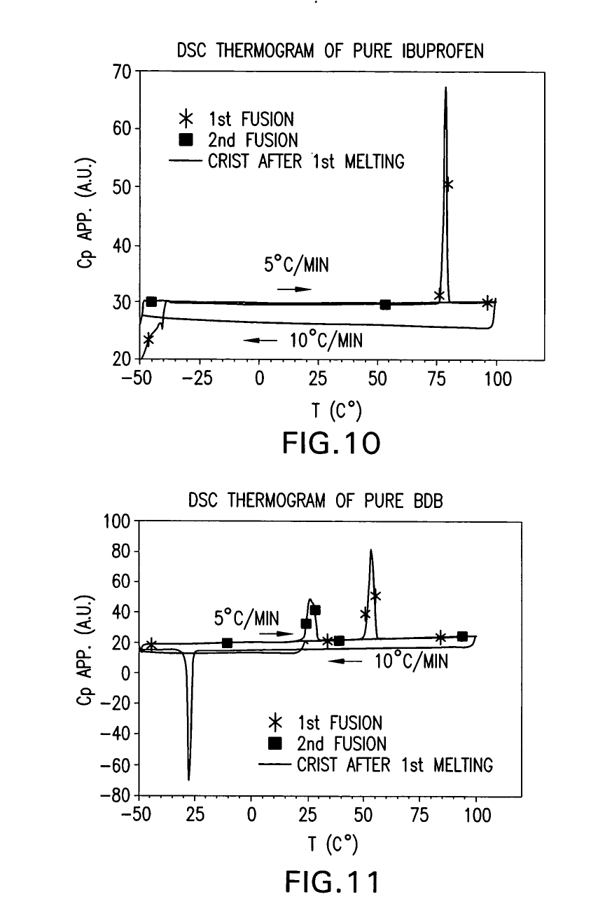

The DSC thermogram of pure ibuprofen (IBU) is presented in FIG. 10. In FIG. 10, the vertical axis is Apparent Specific Heat (C.p), expressed in arbitrary units (A.U.); the horizontal axis is Temperature (in degree Celsius ° C.). The data points for first fusion cycle are presented as crosses, the data points for second fusion cycle are presented as squares, and the data points for cooling cycle are presented as plain line. Powdered IBU sample showed a sharp melting peak at about 77° C. during the first DSC heating cycle at 5° C./min. The subsequent cooling cycle presented no visible thermal event, hence demonstrating the irreversibility of the melting of IBU, or, at least, that IBU presents a significant delay in crystallization. The latter hypothesis was invalidated by the second heating cycle, which showed no thermal event.

The DSC thermogram of pure BDB is presented in FIG. 11. In FIG. 11, the vertical axis is Apparent Specific Heat (C.p), expressed in arbitrary units (A.U.); the horizontal axis is Temperature (in degree Celsius ° C.). The data points for first fusion cycle are presented as crosses, the data points for second fusion cycle are presented as squares, and the data points for cooling cycle are presented as plain line. Powdered BDB sample showed a sharp melting peak at about 51° C. during the first DSC heating cycle at 5° C./min. The subsequent cooling cycle presented a single sharp crystallization peak at about −26.0° C. A second heating in the same conditions than first one has shown an endothermic peak about at room T at Tₒₙₛ=23.3° C. corresponding to the melting of the crystalline variety formed at −26° C.

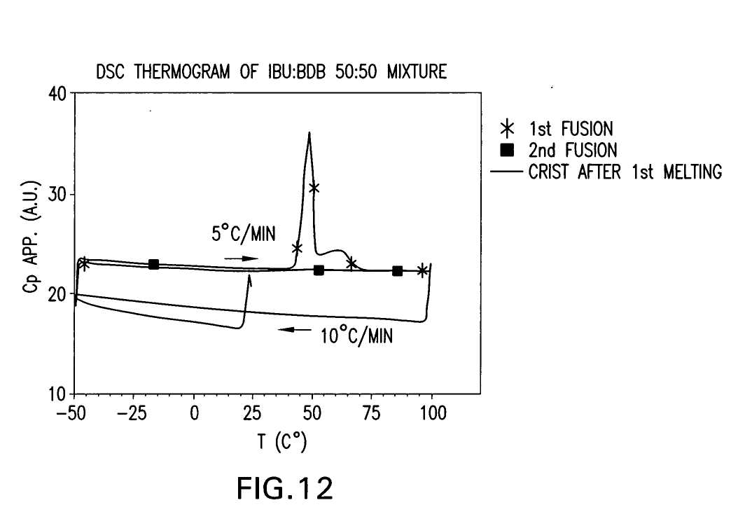

A IBU:BDB 50:50 mixture was prepared and analyzed for DSC. See FIG. 12, wherein the vertical axis is Apparent Specific Heat (C.p), expressed in arbitrary units (A.U.); the horizontal axis is Temperature (in degree Celsius ° C.); and wherein the data points for first fusion cycle are presented as crosses, the data points for second fusion cycle are presented as squares, and the data points for cooling cycle are presented as plain line. The DSC thermogram obtained upon first heating of the mixture shows a rather broad endotherm at about 42-43° C. (corresponding to melting of BDB) followed by a broad endotherm (corresponding to melting of IBU) which extends to about 70° C. Remarkably, temperatures of endotherm (i.e. melting) are lowered by about 5° C. and 27° C. respectively, and both endotherms are broadened. This means that both compounds interact each other to co-solubilize into the melted liquid. More particularly, this clearly shows depression of IBU melting point in presence of BDB. Interestingly the IBU:BDB mixture does re-crystallize neither upon subsequent cooling nor during the following second heating cycle. No first order or second order (glass transition) event is observed on cooling or subsequent heating.

A IBU:BDB 70:30 mixture was prepared and analyzed for DSC. It displays a thermal behavior intermediate between that of pure IBU and that of IBU:BDB 50:50 mixture studied beforehand. Similarly, a IBU:BDB 30:70 mixture was prepared and analyzed for DSC, and displays a thermal behavior intermediate between that of pure BDB and that of IBU:BDB 50:50 mixture. More particularly, data of melting enthalpy suggests that IBU starts to solubilize into melt BDB. Then, once the mixture of both compounds is formed by co-solubilization and/or melting, it does not re-crystallize upon subsequent cooling.

All the examples aforementioned demonstrate that addition of BDB to IBU does alter the thermal events (melting and crystallization) of IBU. More particularly, addition of BDB does enhance supercooling (or delay in crystallization) observed for IBU, by forming a new intimate mixture which displays lowered melting point.

Therefore all the examples aforementioned demonstrate the benefit of adding specific amount of CFI to NSAID in order to decrease melting point of said NSAID.

### Example 2.2

### Aqueous Blending of Bulk Powdered CFI and NSAID

Various mixtures of NSAID and CFI, with the ratio of API to CFI ranging from 90:10 to 10:90, were prepared as described in Example 1.2. The samples were then checked visually. Surprisingly, some mixtures of NSAID and CFI were maintained as stable transparent droplets.

For instance, compositions containing 1% ibuprofen and BDB at ratios from 80:20 and below were still visually stable (presence of oil droplets) at ambient temperature. (Noteworthy, pure ibuprofen and IBU:BDB 90:10 mixture formed a solid off-white agglomerate upon cooling). Droplets were easily re-suspended by gentle hand-shaking. A small aliquot of the re-constituted emulsion showed no crystallization when checked microscopically. For illustration, ibuprofen 1% w/w can be solubilized in a hydro-alcoholic mixture containing at least about 50% by weight of the total mixture of ethanol.

Similarly, compositions containing 1% ketoprofen and PES at ratios from 40:60 and below were still visually stable (presence of oil droplets) after several months at ambient temperature. Droplets were easily re-suspended by gentle hand-shaking. A small aliquot of the re-constituted emulsion showed no crystallization when checked microscopically. Formulations containing ketoprofen and PES in ratios superior or equal to about 50:50 (up to 100:00, i.e. in absence of PES) did all present solid drug particles more or less rapidly. Noteworthy, the higher the proportion of PES in the mixture is, the longer it took to observe presence of such particles, i.e. ratio 90:10 precipitated before ratio 80:20, which precipitated before ratio 70:30, etc. until ratio 50:50. For illustration, solubilization of ketoprofen 1% w/w would require about a hydro-alcoholic mixture containing at least 40% by weight of the total mixture of ethanol. However, ibuprofen or ketoprofen would crystallize massively upon—quick—evaporation of ethanol, hence potentially impairing skin permeation.

In view of the foregoing, it is demonstrated that alcohol-free semi-solid formulations of NSAID wherein NSAID are present under an amorphous, liquid state can easily be achieved by the use of the present invention, i.e. by decreasing melting point of NSAID thanks to a chemical fragrance ingredient.

Various mixtures of ketoprofen and PES (ratio of API to CFI ranging from 90:10 to 50:50) were prepared as described in Example 1.2, except that 10% of the water was replaced by ethanol. The samples were then checked visually. Surprisingly, all mixtures of ketoprofen and PES were maintained as stable transparent droplets. A small aliquot of the re-constituted emulsion showed no crystallization when checked microscopically after several months at room temperature. Noteworthy, 1% ketoprofen in a hydro-alcoholic mixture consisting in 10% of ethanol and 89% of water (“blank” reference) did present solid particles of ketoprofen within a few hours.

This suggests that addition of a very low amount of ethanol (10% w/w or less), wherein said amount is not high enough to allow solubilization of ketoprofen, can further stabilize ketoprofen:PES oil droplets, and thereby enables to increase the ratio of ketoprofen to PES.

In view of the foregoing, it is demonstrated that addition of a low amount of ethanol to a API:CFI mixture, wherein said amount of ethanol is not able to solubilize totally ketoprofen, can further stabilize API:CFI oil droplets. Benefit of incorporating such small amounts of ethanol (typically not more than 30% w/w, preferably not more than 20% w/w, more preferably not more than 10% w/w, and even more preferably as less as possible) therefore allows for increasing the ratio of API to CFI, whilst still obtaining a substantially alcohol-free stable emulsion of NSAID with a pleasant odor profile.

It will appear obvious to the man of the art that ethanol can be replaced in part or inn totality by another short-chain alcohol, such as, but not limited to, propanol, isopropanol or butanol.

### Example 2.3

### Semi-Solid Gel Composition of Amorphous, Non-Solid NSAID

Droplets of NSAID:CFI obtained by practice of the present invention can be finely divided by gentle mixing and then suspended homogeneously thanks to the use of emulsifiers and/or gelling agents known to the one skilled in the art of compounding pharmaceutical products.

Following exemplary formulations comprise should not be interpreted as limitative, and variations may appear obvious to the skilled artisan.

### Example 2.3.1

### Ibuprofen Emulgel

5.0 g of ibuprofen and 1.0 g of phenyl ethyl salicylate are heated together until formation of transparent oil. Oil is then incorporated in a carbomer dispersion consisting in 2.0 g of carbomer C980NF, 10.0 g propylene glycol, and 47.7 g of purified water. A diisopropylamine solution (5.5 g of diisopropylamine in 28.8 g of ethanol) is then added under vigorous mechanical stirring into the carbomer dispersion. A white, homogeneous, opalescent creamy gel is then formed upon neutralization of carbomer. Microscopic examination (STEMI 2000C microscope, Carl Zeiss, Germany) reveals absence of drug crystals.

The obtained gel presents a pleasant balsamic, floral, rose fragrance note.

### Example 2.3.2

### Ibuprofen Emulgel

Same as in Example 2.3.1 except that PES is absent.

### Example 2.3.3

### Ketoprofen Emulgel

2.5 g of ketoprofen and 0.83 g of phenyl ethyl salicylate are heated together until formation of a transparent oil. Carbomer copolymer Type B (PEMULEN TR1 NF, Noveon, Ohio, USA) is then incorporated into the cooled oil and mixed for about 15 minutes. In parallel, a carbomer dispersion consisting in 0.3 g of carbomer C980NF and 80 g of purified water is prepared. The oil phase is then thoroughly introduced into the carbomer dispersion under gentle stirring and homogenized for about 30 minutes. It is then neutralized by a 25% w/w triethanolamine aqueous solution until reaching a pH of about 5.4. A white, homogeneous, opalescent creamy gel is then formed. Microscopic examination (STEMI 2000C microscope, Carl Zeiss, Germany) reveals absence of drug crystals.

The obtained gel presents a pleasant balsamic, floral, rose fragrance note.

### Example 2.4

### In Vitro Skin Permeation Studies

### Example 2.4.1

### In Vitro Skin Permeation of an Ibuprofen Emulgel

Formulations disclosed in Examples 2.3.1 and 2.3.2 were compared for in vitro skin permeation. Table 5 herein after presents exemplary components of ibuprofen gel formulations used in the following experiments.

TABLE 5
Composition of Formulations (% w/w)
FORMULATION

Denomination

| | Example 2.3.1 | Example 2.3.2 |
|---|---|---|
| Composition | % w/w | % w/w |
| Ibuprofen | 5.00 | 5.00 |
| Propylene glycol | 10.0 | 10.0 |
| Phenyl ethyl salicylate | — | 1.00 |
| Carbomer (Carbopol C980NF) | 2.00 | 2.00 |
| Diisopropylamine | 5.50 | 5.50 |
| Ethanol | 28.8 | 28.8 |
| Purified water | 48.7 | 47.7 |
| Total | 100.00 | 100.00 |

Fresh sliced pig ear skin was used for the permeation studies using Franz cells as described in section “B—In Vitro Skin Permeation Methodology”.

Transdermal delivery of ibuprofen using formulations exemplified in Table 5 herein above was assessed as described in Example 1.4. The results presented in Table 6 show the mean values of cumulative delivered amount of ibuprofen after 24 hours.

TABLE 6
Ibuprofen Cumulative Delivery After 24 hours Permeation

| | N (number | Time | Mean Cumulative |
|---|---|---|---|
| Formulation | of samples) | (in hours) | Delivery (μg/cm²) |
| 2.3.1 | 4 | 24 | 27.607 |
| 2.3.2 | 4 | 24 | 19.328 |

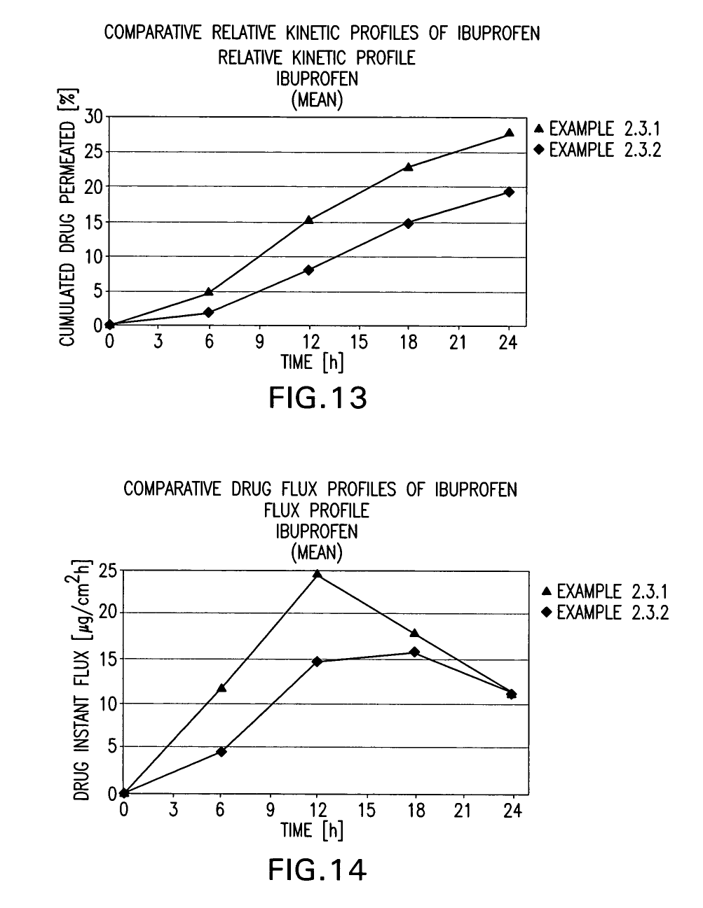

The relative kinetic delivery profiles of ibuprofen over the 24 hour permeation are presented in FIG. 13. In FIG. 13, the vertical axis is Cumulated Drug Permeated (μg/cm²), the horizontal axis is Time (in hours). Further, the transdermal flux profiles of ibuprofen over the 24 hour permeation are presented in FIG. 14. In FIG. 14, the vertical axis is Flux (μ/cm²/hr), the horizontal axis corresponds to sampling times (in hours). The data points for Formulation 2.3.1 are presented as upright triangles, and the data points for Formulation 2.3.2 are presented as diamonds.

The data presented in Table 6 and FIGS. 13 and 14 illustrate the surprising discovery that ibuprofen:PES mixtures of the present invention allows for a skin permeation enhancement of ibuprofen. A huge increase (+43%) in transdermal in vitro bioavailability was observed (from about 19.3% to about 27.6%) when formulating ibuprofen as an amorphous oil with PES.

In view of the foregoing, it is demonstrated that decrease of melting point of NSAID by CFI provides a method to enhance transdermal or transmucosal skin permeation of said NSAID, without having the need to recourse to the use of high level of organic solvent such as ethanol.

### Example 2.4.2

### In Vitro Skin Permeation of a Ketoprofen Emulgel

Formulation disclosed in Examples 2.3.3 is compared to a ketoprofen marketed drug product reference (KETUM®) for in vitro skin permeation. Table 7 herein after presents exemplary components of ketoprofen gel formulations used in the following experiments.

TABLE 7
Composition of Formulations (% w/w)
FORMULATION

Denomination

| | |
|---|---|
| KETUM ® | Example 2.3.3 |

pH

| | 5.5 | 5.4 |
|---|---|---|
| Composition | % w/w | % w/w |
| Ketoprofen | 2.50 | 2.50 |
| Phenyl ethyl salicylate | — | 0.83 |
| Carbomer | 1.50* | 0.50** |
| Ethanol | 40 ml/g | — |
| Diethanolamine | 1.35 | |
| Triethanolamine | — | 0.1875 |
| Lavender oil | 0.1 ml/g | |
| Purified water | q.s. 100.0 | q.s. 100.0 |

*Type of carbomer used in KETUM ® is unknown
**0.2% PEMULEN TR1NF and 0.3% C980NF

Fresh sliced pig ear skin was used for the permeation studies using Franz cells as described in section “B—In Vitro Skin Permeation Methodology”.

Transdermal delivery of ketoprofen using formulations exemplified in Table 7 herein above was assessed as described in Example 1.4. The results presented in Table 8 show the mean values of cumulative delivered amount of ketoprofen after 24 hours.

TABLE 8
Ketoprofen Cumulative Delivery After 24 hours Permeation

| | N (number | Time | Mean Cumulative |
|---|---|---|---|
| Formulation | of samples) | (in hours) | Delivery (μg/cm²) |
| KETUM ® | 4 | 24 | 3.397 |
| 2.3.3 | 4 | 24 | 5.285 |

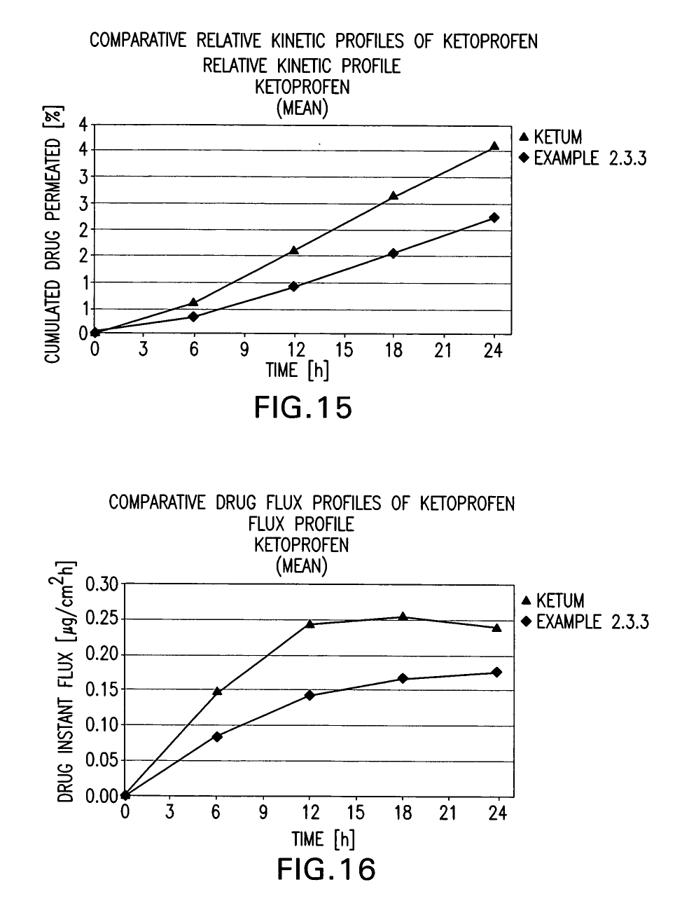

The relative kinetic delivery profiles of ketoprofen over the 24 hour permeation are presented in FIG. 15. In FIG. 15, the vertical axis is Cumulated Drug Permeated (μg/cm²), the horizontal axis is Time (in hours). Further, the transdermal flux profiles of ketoprofen over the 24 hour permeation are presented in FIG. 16. In FIG. 16, the vertical axis is Flux (μg/cm²/hr), the horizontal axis corresponds to sampling times (in hours). The data points for KETUM® are presented as diamonds, and the data points for formulation 2.3.3 are presented as square.

The data presented in Table 8 and FIGS. 15 and 16 illustrate the surprising discovery that ketoprofen:PES mixtures of the present invention allows for a skin permeation enhancement of ketoprofen. Example 2.3.3 exhibits a 61.3% greater cumulated ketoprofen permeated amount over a 24-hour period compared to the hydroalcoholic gel KETUM® (3.58% versus 2.22%, respectively). As shown in FIG. 16, the maximum drug flux is also higher for the formulation of Example 2.3.3 than for KETUM® (0.25 microgram/cm²h maximum drug instant flux versus 0.17 microgram/cm²h, respectively), representing a 47% improvement. Thus, it was surprisingly found that an ethanol-free composition comprising ketoprofen-PES mixture in water (more than 90% w/w of the total composition) exhibits greater skin permeation than a marketed reference drug product.

In view of the foregoing, it is demonstrated that decrease of melting point of NSAID by CFI provides a method to enhance transdermal or transmucosal skin permeation of said NSAID, without having the need to recourse to the use of high level of organic solvent such as ethanol.

### Example 3

### Melting Point Depression Effects of CFI on Anticholinergic Drugs

Melting point depression effects of CFI on anticholinergic drugs were investigated. OXY (OXY) free base was selected as the anticholinergic drug model.

### Example 3.1

### Dry Blending of Bulk Powdered CFI and Oxybutynin

Differential scanning calorimetry (DSC) thermal analyses on OXY:CFI mixtures were conducted as described in Example 1.1 herein above.

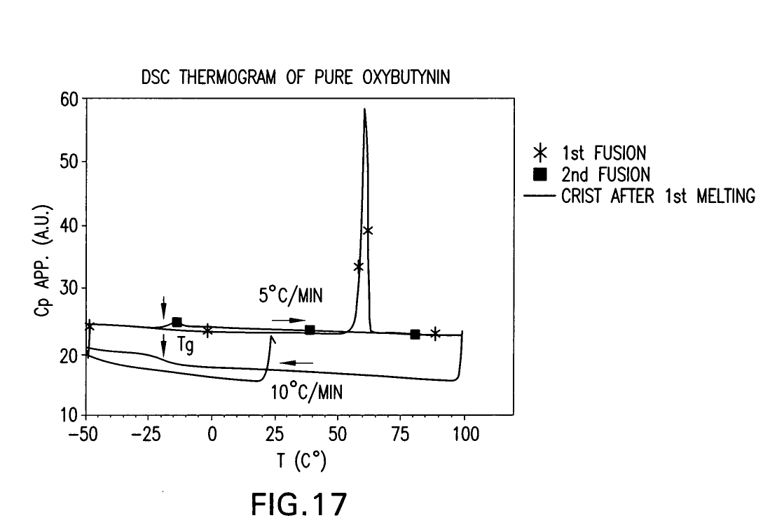

The DSC thermogram of pure OXY is presented in FIG. 17. In FIG. 17, the vertical axis is Apparent Specific Heat (C.p), expressed in arbitrary units (A.U.); the horizontal axis is Temperature (in degree Celsius ° C.). The data points for first fusion cycle are presented as crosses, the data points for second fusion cycle are presented as squares, and the data points for cooling cycle are presented as plain line. Powdered OXY sample showed a sharp melting peak at about 58.5° C. during the first DSC heating cycle at 5° C./min. The subsequent cooling cycle presented no visible first order thermal event. However a second order thermal event (glass transition) was observed at −20° C., hence demonstrating that melting is not reversible or at least that crystallization is delayed. Upon second heating cycle, the glass transition is followed by a small relaxation peak ending at about −6° C.

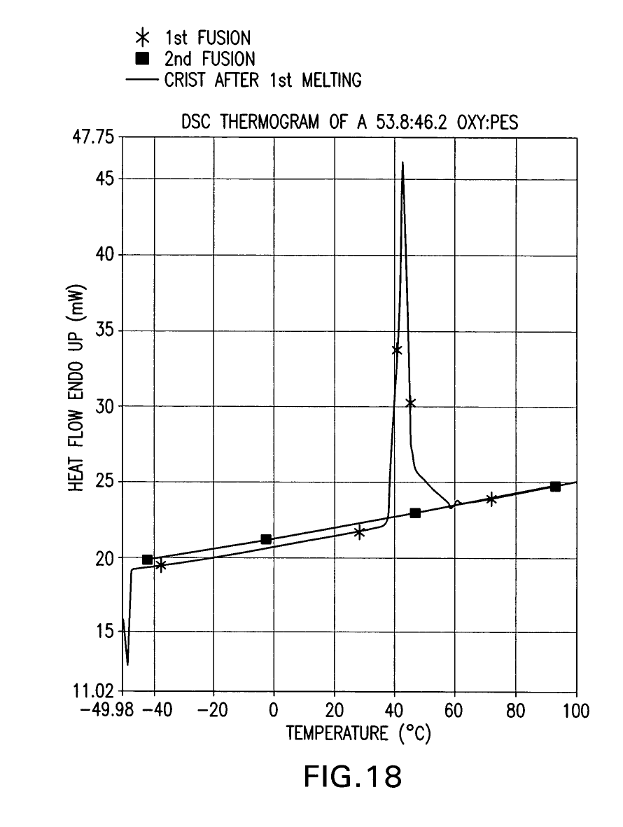

A OXY:PES 53.8:46.2 mixture was prepared and analyzed for DSC. See FIG. 18, wherein the vertical axis is Apparent Specific Heat (C.p), expressed in arbitrary units (A.U.); the horizontal axis is Temperature (in degree Celsius ° C.); and wherein the data points for first fusion cycle are presented as crosses, the data points for second fusion cycle are presented as squares, and the data points for cooling cycle are presented as plain line. The DSC thermogram obtained upon first heating of the mixture shows a sharp broad endotherm at about 38-45° C. (corresponding to melting of PES) followed by a broad endotherm (corresponding to melting of OXY) which extends to about 60° C. Both compounds interact with each other and co-solubilize into melted liquid. Mixture does re-crystallize neither upon subsequent cooling nor during the following second heating. However, a second order thermal event (glass transition) is observed at about −45° C. both upon cooling or during second heating cycle.

A OXY:PES 70:30 mixture was prepared and analyzed for DSC. It displays a thermal behavior intermediate between that of pure OXY and that of OXY:IBU mixture studied beforehand with a ratio close to 50:50. Similarly, a OXY:PES 30:70 mixture was prepared and analyzed for DSC, and displays a thermal behavior intermediate between that of pure PES and that of OXY:IBU mixture with a ratio close to 50:50. More particularly, data of melting enthalpy suggests that PES starts to solubilize into melt OXY. Then, once the mixture of both compounds is formed by co-solubilization and/or melting, it does not re-crystallize upon subsequent cooling.

All the examples aforementioned demonstrate that addition of PES to OXY does alter the thermal events (melting and crystallization) of OXY. More particularly, addition of PES does enhance supercooling (or delay in crystallization) and glass transition observed for OXY, by forming a new intimate mixture which displays lowered melting point.

Therefore all the examples aforementioned demonstrate the benefit of adding specific amount of CFI to NSAID in order to decrease melting point of said NSAID.

### Example 3.2

### Aqueous Blending of Bulk Powdered CFI and Oxybutynin

Various mixtures of OXY and CFI, with the ratio of API to CFI ranging from 90:10 to 10:90, were prepared as described in Example 1.2. The samples were then checked visually. A “blank” sample (i.e. not containing PES) demonstrated precipitation of crystallized OXY. Surprisingly, some mixtures of OXY and CFI were maintained as stable transparent droplets. For instance, some compositions (OXY:PES ratios from 50:50 and below) containing about 1% w/w of OXY:PES mixtures were still visually stable (presence of oil droplets) at ambient temperature after 24 hours. However, droplets were not easily re-suspended by gentle hand-shaking and exhibit a slight tendency to “stick” to the glass vials. This phenomenon can be reduced or even prevented by addition of low amounts of ethanol. For instance, a composition containing OXY 3.0%, PES 3.0%, ethanol 30%, purified water 64% presents small transparent droplets easily re-suspended by gentle hand shaking (a “control” not containing PES presents crystallized OXY after only a very few days). A small aliquot of the re-constituted emulsion showed no crystallization when checked microscopically: OXY is therefore surprisingly maintained under an amorphous form.

For illustration, OXY free base 3.00% w/w can be solubilized in a hydro-alcoholic mixture containing at least about 55% by weight of the total mixture of ethanol. However, OXY crystallizes massively upon—quick—evaporation of ethanol, hence potentially impairing skin permeation.

### Example 3.3

### Semi-Solid Gel Composition of Amorphous, Non-Solid Anticholinergic Drugs

Droplets of OXY:CFI obtained by practice of the present invention can be finely divided by gentle mixing and then suspended homogeneously thanks to the use of emulsifiers and/or gelling agents known to the one skilled in the art of compounding pharmaceutical products.

For illustration, one can consider the following representative formulations (percents are expressed in percent weight based on the weight of the formulation):

| | |
|---|---|
| Oxybutynin | 0.1%-5% |
| PES | 0.1%-5% |
| Ethanol | 5%-40% |
| TRANSCUTOL | 2.5%-15% |
| PEMULEN TR1 NF | 0.05%-0.5% |
| C981 or ETD2020 | 0.2%-1% |
| Buffering agent | qs pH 4.5-8.5 |
| Purified water | qs 100 |

### Example 4

### Melting Point Depression Effects of CFI on Antinociceptive Drugs

Melting point depression effects of CFI on antinociceptive drugs were investigated. Fentanyl free base was selected as the anticholinergic drug model.

### Example 4.1

### Aqueous Blending of Bulk Powdered CFI and Fentanyl

Various mixtures of fentanyl and CFI, with the ratio of API to CFI ranging from 90:10 to 10:90, were prepared as described in Example 1.2. The samples were then checked visually. A “blank” sample (i.e. not containing PES) demonstrated precipitation of crystallized fentanyl. Surprisingly, some mixtures of fentanyl and CFI were maintained as stable transparent droplets.

For instance, some compositions (fentanyl:PES ratios from 40:60 and below) containing about 1% w/w of fentanyl:PES mixtures were still visually stable (presence of oil droplets) at ambient temperature after 24 hours. A small aliquot of the re-constituted emulsion showed no crystallization when checked microscopically: fentanyl is surprisingly maintained under an amorphous form. Mixtures of fentanyl and PES of the present invention can be further stabilized and enriched in fentanyl thanks to the addition of low amounts of ethanol, wherein the amount of ethanol is not responsible for the total solubilization of fentanyl. For instance, a composition containing 1% fentanyl, 1% PES, 15% ethanol and 83% purified water prepared as described in § 1.2 presents stable oil droplets of amorphous fentanyl.

For illustration, fentanyl 1% w/w can be solubilized in a hydro-alcoholic mixture containing at least about 40% by weight of the total mixture of ethanol. However, fentanyl would crystallize massively upon—quick—evaporation of ethanol, hence potentially impairing skin permeation.

### Example 4.2

### Semi-Solid Gel Composition of Amorphous, Non-Solid Antinociceptive Drugs

Droplets of fentanyl:CFI obtained by practice of the present invention can be finely divided by gentle mixing and then suspended homogeneously thanks to the use of emulsifiers and/or gelling agents known to the one skilled in the art of compounding pharmaceutical products.

For illustration, one can consider the following additional formulations (percents are expressed in percent weight based on the weight of the formulation):

| | |
|---|---|
| Fentanyl | 0.1%-5% |
| PES | 0.1%-5% |
| Isopropanol | 5%-30% |
| Propylene glycol | 5%-20% |
| MONTANOV 68 | 1%-10% |
| SIMULGEL PHA 600 | 1%-5% |
| Buffering agent | qs pH 4.5-8.5 |
| Purified water | qs 100 |

### Example 5

### Melting Point Depression Effects of CFI on Cardiovascular Drugs

Melting point depression effects of CFI on cardiovascular drugs were investigated. Carvedilol free base (CAR) and clonidine free base (CLO) were selected as cardiovascular drug models.

### Example 5.1

### Aqueous Blending of Bulk Powdered CFI and Carvedilol

Various mixtures of cardiovascular drugs and CFI, with the ratio of API to CFI ranging from 90:10 to 10:90, were prepared as described in Example 1.2. The samples were then checked visually. “Blank” samples (i.e. not containing PES) demonstrated precipitation of crystallized CAR or non melting of clonidine. Surprisingly, some mixtures of CAR and CFI were maintained as stable transparent droplets for about 24 to 48 hours. Even more surprisingly, despite melting point of CLO is close to 130° C. (hence higher than the about 100° C. reached in the water bath), some mixtures of CLO and CFI (CLO:PES 30:70, 20:80 and 10:90) were also maintained as stable transparent droplets for about 24 to 48 hours. A small aliquot of the re-constituted emulsion showed no crystallization when checked microscopically: CAR and CLO are surprisingly maintained under an amorphous form. However crystallization of CAR and CLO occurred ultimately in all vials afterwards.

Droplets of CAR:PES were not easily re-suspended by gentle hand-shaking and exhibit a slight tendency to “stick” to the glass vials. This phenomenon can be reduced or even prevented by addition of low amounts of ethanol, wherein the amount of ethanol is not responsible for the total solubilization of CAR. For instance, a composition containing 1% CAR, 1% PES, 30% ethanol and 68% purified water prepared as described in § 1.2 presents stable oil droplets of amorphous CAR. Similarly, a composition containing 0.5% CLO, 0.5% PES, 10% ethanol and 89% purified water prepared as described in § 1.2 presents stable oil droplets of amorphous CLO (control formulation, i.e. without PES, presented non melted CLO). Same observation was made with a composition containing 0.5% CLO, 0.5% PES, 5% ethanol and 94% purified water prepared as described in § 1.2 presents stable oil droplets of amorphous CLO (control formulation, i.e. without PES, presented non melted CLO).

For illustration, CAR 1% w/w can be solubilized in a hydro-alcoholic mixture containing at least about 80% by weight of the total mixture of ethanol. However, CAR would crystallize massively upon—quick—evaporation of ethanol, hence potentially impairing skin permeation. Similarly, CLO 0.5% w/w can be solubilized in a hydro-alcoholic mixture containing at least about 50% by weight of the total mixture of ethanol.

### Example 5.2

### Semi-Solid Gel Composition of Amorphous, Non-Solid Cardiovascular Drugs

Droplets of CAR:CFI or CLO:CFI obtained by practice of the present invention can be finely divided by gentle mixing and then suspended homogeneously thanks to the use of emulsifiers and/or gelling agents known to the one skilled in the art of compounding pharmaceutical products.

For illustration, one can consider the following additional formulations (percents are expressed in percent weight based on the weight of the formulation):

| | |
|---|---|
| Clonidine | 0.1%-5% |
| PES | 0.1%-5% |
| Ethanol | 5%-40% |
| LABRASOL | 5%-15% |
| Cellulose | 1%-10% |
| Buffering agent | qs pH 4.5-8.5 |
| Purified water | qs 100 |

### Example 6

### Melting Point Depression Effects of CFI on Antiparkinson Drugs

Melting point depression effects of CFI on antiparkinson drugs were investigated. Ropinirole free base (ROP, melting point 65° C.-66° C.) and pramipexole free base (PRA, melting point: 266-270° C.) were selected as the antiparkinson drug models.

### Example 6.1

### Aqueous Blending of Bulk Powdered CFI and Antiparkinson Drugs

Various mixtures of ROP and CFI, with the ratio of API to CFI ranging from 90:10 to 10:90, were prepared as described in Example 1.2. The samples were then checked visually. A “blank” sample (i.e. not containing PES) demonstrated sedimentation of a thick “paste” which could not be easily re-dispersed, therefore jeopardizing formulation of an homogeneous emulsified system. Surprisingly, some mixtures of ROP and CFI were maintained as stable droplets easily re-suspensed by gentle hand shaking.

For instance, the richer in PES the compositions containing about 1% w/w of ROP:PES mixtures are, the easier the re-dispersion of droplets is, and consequently the easier the formulation of a physically stable, homogeneous emulsion is. Small aliquots of re-constituted emulsions showed no crystallization when checked microscopically: ROP is surprisingly maintained under an amorphous form. The ease of emulsification of the ROP:PES mixture is even further enhanced by addition of low amounts of ethanol, wherein the amount of ethanol is not responsible for the total solubilization of ROP. For instance, a composition containing 1% ROP, 1% PES, 15% ethanol and 83% purified water prepared as described in § 1.2 present stable oil droplets of amorphous ROP easily re-dispersible.

For illustration, ROP 1% w/w requires a hydro-alcoholic mixture containing at least about 40% by weight of the total mixture of ethanol for being totally solubilized.

Similarly, a composition containing 1% PRA, 1% PES, 30% ethanol and 68% purified water prepared as described in § 1.2 present stable oil droplets of amorphous PRA easily re-dispersible. Small aliquots of re-constituted emulsions showed no crystallization when checked microscopically: PRA is surprisingly maintained under an amorphous form.

### Example 6.2

### Semi-Solid Gel Composition of Amorphous, Non-Solid Antiparkinson Drugs

Droplets of antiparkinson drugs:CFI obtained by practice of the present invention can be finely divided by gentle mixing and then suspended homogeneously thanks to the use of emulsifiers and/or gelling agents known to the one skilled in the art of compounding pharmaceutical products.

For illustration, one can consider the following additional formulations (percents are expressed in percent by weight based on the weight of the formulation):

| | |
|---|---|
| Ropinirole | 0.1%-5% |
| PES | 0.1%-5% |
| Antioxidant | 0.01%-1% |
| Ethanol | 5%-30% |
| PEMULEN TR1 NF | 0.05%-0.5% |
| C981 or ETD2020 | 0.2%-1% |
| Buffering agent | qs pH 4.5-8.5 |
| Preservatives | 0.01%-2% |
| Purified water | qs 100 |

### Example 7

### Melting Point Depression Effects of CFI on Sexual Hormones

Melting point depression effects of CFI on sexual hormones were investigated. Testosterone (TES) was selected as the vasoconstrictor drug model.

### Example 7.1

### Aqueous Blending of Bulk Powdered CFI and Testosterone

Various mixtures of sexual hormones and CFI, with the ratio of API to CFI ranging from 90:10 to 10:90, were prepared as described in Example 1.2. The samples were then checked visually. A “blank” sample (i.e. not containing PES) demonstrated non melting of sexual hormones. Surprisingly, despite melting point of TES close to 155° C. (hence higher than the about 100° C. reached in the water bath), some mixtures of TES and CFI (TES:PES 40:60, 30:70, 20:80 and 10:90) were also maintained as stable transparent droplets once cooled down to room temperature. A small aliquot of the re-constituted emulsion showed no crystallization when checked microscopically: TES is surprisingly maintained under an amorphous form. However crystallization of TES occurred ultimately in all the vials.

Stability of TES:PES mixtures can be further enhanced by addition of low amounts of ethanol, wherein the amount of ethanol is not responsible for the total solubilization of TES. For instance, a composition containing 0.5% TES, 0.5% PES, 15% ethanol and 84% purified water prepared as described in § 1.2 presents stable oil droplets of amorphous TES (control formulation, i.e. without PES, demonstrated non melting of TES). Another composition containing 0.5% TES, 0.5% PES, 30% ethanol and 69% purified water prepared as described in § 1.2 presents also stable oil droplets of amorphous TES. (control formulation, i.e. without PES, demonstrated extensive precipitation and crystallization of TES within a few seconds though being a clear solution when tested (probably because heat increased testosterone solubility in purified water).

For illustration, TES 0.5% w/w can be solubilized in a hydro-alcoholic mixture containing at least about 50% by weight of the total mixture of ethanol. However, TES crystallizes massively upon—quick—evaporation of ethanol, hence potentially impairing skin permeation.

### Example 7.2

### Semi-Solid Gel Composition of Amorphous, Non-Solid Sexual Hormones

Droplets of sexual hormones:CFI obtained by practice of the present invention can be finely divided by gentle mixing and then suspended homogeneously thanks to the use of emulsifiers and/or gelling agents known to the one skilled in the art of compounding pharmaceutical products.

For illustration, one can consider the following additional formulations (percents are expressed in percent by weight based on the weight of the formulation):

| | |
|---|---|
| Testosterone | 0.1%-5% |
| PES | 0.1%-5% |
| Lauroglycol | 5%-15% |
| LABRASOL | 10%-45% |
| TRANSCUTOL | 5%-15% |
| Ethanol | 5%-30% |
| Chelating agent | 0.01%-1% |
| Preservatives | 0.01%-2% |
| Purified water | qs 100 |

### Example 8

### Melting Point Depression Effects of CFI on Anti Acne Drugs

Melting point depression effects of CFI on anti acne drugs were investigated. An anti androgenically active compound (X) disclosed in U.S. Pat. No. 6,875,438, with a melting point of about 101° C., was selected as the anti acne drug model.

### Example 8.1

### Aqueous Blending of Bulk Powdered CFI and Testosterone

Various mixtures of X and CFI, with the ratio of API to CFI ranging from 90:10 to 10:90, were prepared as described in Example 1.2. The samples were then checked visually. A “blank” sample (i.e. not containing PES) demonstrated non melting of anti acne drug. Surprisingly, some mixtures of X and CFI (X:PES 50:50 and below) were maintained as stable transparent droplets for several days at ambient temperature. A small aliquot of the re-constituted emulsion showed no crystallization when checked microscopically: X is surprisingly maintained under an amorphous form.

Stability of X:PES mixtures can be further enhanced by addition of low amounts of ethanol, wherein the amount of ethanol is not responsible for the total solubilization of TES. For instance, a composition containing 1% X, 1% PES, as little as 5% ethanol and 83% purified water prepared as described in § 1.2 presents stable oil droplets of amorphous X.

For illustration, X 1% w/w can be solubilized in a hydro-alcoholic mixture containing at least about 70% by weight of the total mixture of ethanol. However, X crystallizes massively upon—quick—evaporation of ethanol, hence potentially impairing skin permeation.

### Example 8.2

### Semi-Solid Gel Composition of Amorphous, Non-Solid Anti Acne Drugs

Droplets of sexual hormones:CFI obtained by practice of the present invention can be finely divided by gentle mixing and then suspended homogeneously thanks to the use of emulsifiers and/or gelling agents known to the one skilled in the art of compounding pharmaceutical products. Formulation of substantially alcohol-free compositions of X represents a great benefit for patients suffering from acne, since presence of significant amounts of alcohol would potentially cause further drying, redness, irritation and itching of the damaged, sensitive, acneic skin.

For illustration, one can consider the following additional formulations (percents are expressed in percent by weight based on the weight of the formulation):

| | |
|---|---|
| Anti-acne drug | 0.1%-5% |
| PES | 0.1%-5% |
| Permeation enhancer | 0.1%-5% |
| Lauroglycol | 5%-15% |
| LABRASOL | 10%-45% |
| TRANSCUTOL | 5%-15% |
| Ethanol | 5%-30% |
| Preservatives | 0.01%-2% |
| Silicone | 1%-5% |
| Purified water | qs 100 |

### Example 9

### Melting Point Depression Effects of CFI on Anti-Emetic Drugs

Melting point depression effects of CFI on anti-emetic drugs were investigated. Granisetron free base (GRA) was selected as the anti-emetic drug model.

### Example 9.1

### Aqueous Blending of Bulk Powdered CFI and Granisetron

Various mixtures of GRA and CFI, with the ratio of API to CFI ranging from 90:10 to 10:90, were prepared as described in Example 1.2. The samples were then checked visually. A “blank” sample (i.e. not containing PES) demonstrated non melting of anti-emetic drug. Surprisingly, despite melting point of GRA is close to about 151.5° C. (hence higher than the about 100° C. reached in the water bath), some mixtures of GRA and CFI (namely, GRA:PES 30:70, 20:80 and 10:90) appeared transiently as stable transparent droplets once cooled down to room temperature, thereby witnessing the melting point depressant effect of PES. However crystallization of GRA occurred ultimately in all the vials after a few days. Stability of GRA:PES mixtures can be further enhanced by addition of low amounts of ethanol, wherein the amount of ethanol is not responsible for the total solubilization of TES. For instance, a composition containing 0.5% GRA, 0.5% PES, 30% ethanol and 69% purified water prepared as described in § 1.2 presents stable oil droplets of amorphous GRA, whereas a “control” composition (i.e. not containing PES) demonstrated impossibility to melt GRA at a temperature lower than 100° C. A small aliquot of the re-constituted GRA:PES emulsion confirmed absence of crystallization when checked microscopically: GRA is therefore surprisingly maintained under an amorphous form.

For illustration, GRA 0.5% w/w can be solubilized in a hydro-alcoholic mixture containing at least about 40% by weight of the total mixture of ethanol. However, GRA crystallizes massively upon—quick—evaporation of ethanol, hence potentially impairing skin permeation.

### Example 9.2

### Semi-Solid Gel Composition of Amorphous, Non-Solid Anti-Emetic Drugs

Droplets of anti-emetic drugs:CFI obtained by practice of the present invention can be finely divided by gentle mixing and then suspended homogeneously thanks to the use of emulsifiers and/or gelling agents known to the one skilled in the art of compounding pharmaceutical products.

Following exemplary formulations comprise should not be interpreted as limitative, and variations may appear obvious to the man in the art.

### Example 9.2.1

### Granisetron Emulgel

0.50 g of granisetron base and 0.50 g of phenyl ethyl salicylate are added to 65.0 g of purified water and 30.0 g of ethanol into a sealed container and heated in a water-bath until formation of transparent droplets, and then let cooled down to room temperature. 4.0 g of SIMULGEL PHA 600 (Seppic, Paris, France) are then added under gentle mixing to the granisetron emulsion. A white, homogeneous, opalescent creamy gel is then formed. Microscopic examination (STEMI 2000C microscope, Carl Zeiss, Germany) reveals absence of drug crystals.

The obtained alcohol-free gel presents a pleasant balsamic, floral, rose fragrance note.

### Example 9.2.2

### Granisetron Dispersion

Same as Example 9.2.1 but without PES. A white, macroscopically homogeneous, opalescent creamy gel is then formed. Microscopic examination (STEMI 2000C microscope, Carl Zeiss, Germany) reveals presence of drug crystals.

### Example 9.3

### In Vitro Skin Permeation of a Granisetron Emulgel

Formulations disclosed in Examples 9.2.1 and 9.2.2 were compared for in vitro skin permeation. Table 9 herein after presents exemplary components of granisetron gel formulations used in the following experiments.

TABLE 9
Composition of Formulations (% w/w)

| FORMULATION | | |
|---|---|---|
| Denomination | Example 9.2.1 | Example 9.2.2 |
| Composition | % w/w | % w/w |
| Granisetron | 0.50 | 0.50 |
| Phenyl ethyl salicylate | 0.50 | — |
| Ethanol | 30.0 | 30.0 |
| SIMULGEL PHA 600 | 4.00 | 4.00 |
| Purified water | 65.0 | 65.5 |
| Total | 100.00 | 100.00 |

Fresh sliced pig ear skin was used for the permeation studies using Franz cells as described in section “B—In Vitro Skin Permeation Methodology”.

Transdermal delivery of granisetron using formulations exemplified in Table 9 herein above was assessed as described in Example 1.4. The results presented in Table 10 show the mean values of cumulative delivered amount of granisetron after 24 hours.

TABLE 10
Granisetron Cumulative Delivery After 24 hours Permeation

| | N (number of | Time | Mean Cumulative |
|---|---|---|---|
| Formulation | samples) | (in hours) | Delivery (μg/cm²) |
| 9.2.1 | 4 | 24 | 3.905 |
| 9.2.2 | 3 | 24 | 1.386 |

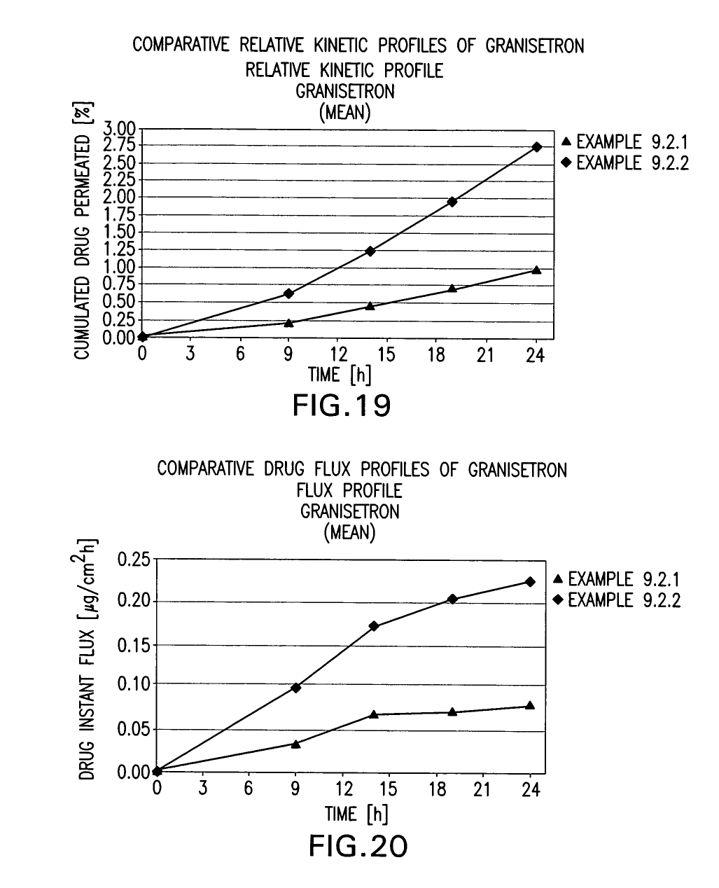

The relative kinetic delivery profiles of granisetron over the 24 hour permeation are presented in FIG. 19. In FIG. 19, the vertical axis is Cumulated Drug Permeated (1 g/cm²), the horizontal axis is Time (in hours). Further, the transdermal flux profiles of granisetron over the 24 hour permeation are presented in FIG. 20. In FIG. 20, the vertical axis is Flux (1 g/cm²/hr), the horizontal axis corresponds to sampling times (in hours). The data points for Formulation 9.2.1 are presented as diamonds, and the data points for Formulation 9.2.2 are presented as upright triangles.

The data presented in Table 10 and FIGS. 19 and 20 illustrate the surprising discovery that granisetron:PES mixtures of the present invention allows for a skin permeation enhancement of granisetron. A huge increase (+182%) in transdermal in vitro bioavailability was observed (from about 0.97% to about 2.74%) when formulating granisetron as an amorphous oil with PES.

In view of the foregoing, it is demonstrated that decrease of melting point of anti-emetic drugs by CFI provides a method to enhance transdermal or transmucosal skin permeation of said anti-emetic drugs, without having the need to use high levels of organic solvent such as ethanol.

It will be apparent to those skilled in the art that various modifications and variations can be made in the method and composition of the present invention without departing from the spirit or scope of the invention. Thus, it is intended that the present invention include modifications and variations that are within the scope of the appended claims and their equivalents.

All patents, publications, and patent applications cited in this specification are herein incorporated by reference as if each individual patent, publication, or patent application was specifically and individually indicated to be incorporated by reference in its entirety for all purposes.

What is claimed is:

1. A pharmaceutical composition comprising:
at least one active agent;
one chemical fragrance or flavor ingredient present in an amount sufficient to act as melting point depressant agent for the at least one active agent;
optionally, a co-melting point depressant agent; and
wherein:
the at least active agent exhibits a depressed melting point and is present as an amorphous liquid or semi-solid;
the melting point depressant agent imparts a pleasant olfactory profile to the composition; and
the amount of the co-melting point depressant agent, when present, is lower than that amount required for total solubilization of the at least one active agent in a similar composition not containing the chemical fragrance or flavor ingredient.

2. The composition of claim 1, wherein the chemical fragrance ingredient is selected from the group including phenyl ethyl salicylate, 4-(1,3-benzodioxol-5-yl)butan-2-one, β-naphtyl isobutyl ether, indeno-m-dioxin tetrahydro, ortho tertiary butyl cyclohexanol, or mixtures thereof.

3. The composition of claim 1, wherein the at least one active agent exhibits a melting point below 250° C.

4. The composition of claim 1, wherein the at least one active agent exhibits a melting point below 150° C.

5. The composition of claim 1, wherein the at least one active agent exhibits a melting point below 100° C.

6. The composition of claim 1, wherein the composition further includes a pharmaceutically acceptable carrier.

7. The composition of claim 6, wherein the pharmaceutically acceptable carrier is substantially hydrophilic.

8. The composition of claim 6, wherein the pharmaceutically acceptable carrier comprises water.

9. The composition of claim 1, wherein the optional co-melting point depressant agent is selected from the group of ethanol, propanol, isopropanol, butanol, propylene glycol, diethylene glycol mono ethyl ether, glycofurol, and mixtures thereof.

10. The composition of claim 6, wherein the pharmaceutically acceptable carrier is alcohol-free.

11. The composition of claim 6, wherein the pharmaceutically acceptable carrier is substantially silicone-based.

12. The composition of claim 9, wherein the composition is in the form of a multiple-phase system including a discontinuous phase and a continuous phase.

13. The composition of claim 12, wherein the discontinuous phase includes the active agent and the chemical fragrance or flavor ingredient, and the continuous phase includes the pharmaceutically acceptable carrier.

14. The composition of claim 1, wherein the composition further includes at least one solvent, permeation enhancer, gelling agent, suspending agent, emulsifying agent, surfactant, co-surfactant, buffering agent, antioxidant, preservative, stabilizer, humectant, colorant, fragrance, flavor, or mixtures thereof.

15. The composition of claim 1, wherein the composition is intended for topical or transdermal administration through the skin or a membrane mucosa

16. The composition of claim 15, wherein the mucosa is the nasal mucosa, the ophthalmic mucosa, the auricular mucosa, the buccal mucosa, the pulmonary mucosa, the gastro-intestinal mucosa, the rectal mucosa, or the vaginal mucosa.

17. The composition of claim 15, wherein the composition is in the form of a gel, lotion, suspension, cream, foam, microemulsion or nanoemulsion, aerosol, spray, patch, bandage, plaster, medicated dressing, capsule.

18. A process to manufacture the composition of claim 1, comprising:
forming a amorphous liquid or semi-solid mixture of at least one active agent and a chemical fragrance or flavor ingredient, and the optional co-melting point depressant agent; and
incorporating said mixture within a pharmaceutically acceptable carrier.

19. The manufacturing process of claim 18, wherein the homogeneous mixture is formed by melting the at least one active agent and the chemical fragrance or flavor ingredient and the optional co-melting point depressant agent altogether.

20. The manufacturing process of claim 18, wherein the homogeneous mixture is formed by simply admixing at ambient temperature the at least active agent and the chemical fragrance or flavor ingredient and the optional co-melting point depressant agent altogether.

21. A method for enhancing transdermal or topical delivery of a pharmacologically active agent through or into the skin or a mucosa, comprising administering to a skin surface or a mucosa of a patient in need thereof a composition of claim 1.
